(19) United States
(12) Patent Application Publication
(10) Pub. No.: US 2019/0298854 A1
(43) Pub. Date: Oct. 3, 2019

(54) DRUG DELIVERY PARTICLES

(71) Applicants: GLAXOSMITHKLINE BIOLOGICALS, SA, Rixensart (BE); Liquidia Technologies, Inc., Morrisville, NC (US)

(72) Inventors: Abdelatif A ELOUAHABI, Rixensart (BE); Patrick POHLHAUS, King of Prussia, PA (US); Laurent Bernard Jean STRODIOT, Rixensart (BE); Ashley GALLOWAY, Morrisville, NC (US); Jin Christiane Lee, Morrisville, NC (US); Michele R. STONE, Morrisville, NC (US)

(73) Assignees: GLAXOSMITHKLINE BIOLOGICALS, SA, Rixensart (BE); Liquidia Technologies, Inc., Morrisville, NC (US)

(21) Appl. No.: 16/082,674

(22) PCT Filed: Mar. 6, 2017
(86) PCT No.: PCT/EP2017/055147

§ 371 Date: Sep. 6, 2018

Related U.S. Application Data
(60) Provisional application No. 62/304,399, filed on Mar. 7, 2016.

(51) Int. Cl. A61K 47/69; A61K 9/16; A61K 47/64; A61K 47/61; A61K 39/08; A61K 39/102

(52) U.S. Cl. CPC A61K 47/6921; A61K 9/1635; A61K 47/6415; A61K 9/1682; A61K 39/08; A61K 39/102; A61K 47/61

(57) ABSTRACT

The present invention concerns drug delivery particles which can prevent interaction between a biologically-active cargo comprised within the particles and components of an aqueous environment in which said particles are present. The particles are sensitive to pH such that above a threshold pH level the biologically-active cargo becomes accessible to the surrounding environment. Such particles are accordingly useful for stably storing a biologically-active cargo in an aqueous composition containing components which would otherwise interact deleteriously with the cargo, and releasing the cargo to mediate a biological effect in the body of an animal, such as a human, to which the composition is administered. Also provided are compositions comprising such particles, as well as methods for making and using such particles and compositions.

### FIELD OF THE INVENTION

The present invention relates to (micro- or nano-) particles which comprise a biologically-active cargo, which cargo is, as a result of its presence within such particles, protected from undesirable interactions with substances co-formulated with the particles such as in a parenteral formulation. The cargo, such as a drug or other therapeutically-relevant agent, can thereby be stably stored in a parenteral formulation, with release of the cargo from the particles occurring only after administration. These particles have particular utility in vaccine compositions.

### BACKGROUND TO THE INVENTION

Many medicinal compositions, such as (therapeutic) drugs or (prophylactic) vaccines, are combination products which contain two or more biologically-active constituents, e.g. active pharmaceutical ingredients or antigens. Such compositions may exhibit a synergistic effect, or may offer advantages such as increased compliance with a treatment regimen e.g. due to a reduced total number of administration, especially in the case of paediatric immunisation schedules.

The respective biologically-active constituents may have associated with them other constituents such as, in the case of vaccines, adjuvants, and the composition as a whole will contain pharmaceutically-acceptable formulation excipients, often in an aqueous formulation. It is known that when co-formulated (i.e. formulated together in a single composition such as a parenteral formulation), one biologically-active constituent may interact with another biologically-active constituent, or with an associated constituent such as an adjuvant or an excipient or even water present in the formulation. Such interaction may have a deleterious impact on the biological effect mediated by at least one of the interacting biologically-active constituents (such impact being ‘deleterious’ relative to the biological effect that such biologically-active constituent would mediate if formulated alone, i.e. as the sole biologically-active constituent).

In the case of vaccines, such deleterious interaction may manifest as a physical or biochemical incompatibility, such as an effect on the stability of the biologically-active constituent, and/or as an in vivo phenomenon adversely impacting on the immune response elicited by the constituent (“immunological interference”). For example in the case of paediatric combination vaccines containing Haemophilus influenzae type b (“Hib”) polysaccharide conjugated to a carrier protein (such as tetanus toxoid, “TT”), together with other antigens adsorbed on aluminium hydroxide adjuvant (such as diphtheria toxoid, tetanus toxoid and acellular pertussis antigens, “DTPa”), the Hib antigen is lyophilised and packaged separately from the liquid, aqueous DTPa/aluminium hydroxide-containing formulation—this is the case in e.g. Infanrix™ Hexa (GSK Vaccines). There are two reasons for this: first, because the Hib-derived polysaccharide part (polyribosylribitol, “PRP”) of the Hib conjugate antigen is labile to hydrolytic degradation when in aqueous formulation (i.e. the Hib undergoes a physical/biochemical interaction with water molecules, reducing its stability); and secondly because the PRP can interact with aluminium hydroxide to form a network of particles (“flocculation”) which is believed to mask PRP epitopes from the recipient's immune system (i.e. the Hib exhibits immunological interference when formulated in the presence of aluminium hydroxide). In the case of vaccines such as Infanrix™ Hexa, partitioning the vaccine components between a liquid, aqueous component and a lyophilised component, which are extemporaneously reconstituted at the time of administration, solves the above problems. However, it leads to a two-part vaccine requiring a reconstitution step to be carried out by the medical personnel administering the vaccine. A one-part liquid vaccine with all components in a single container would offer advantages such as simplified filling/packaging, transport/storage, and administration.

There is a accordingly a desire for solutions to the problem of how to combine physically, biochemically and/or immunologically incompatible constituents of combination (“multivalent”) medicinal compositions into one-part liquid, aqueous compositions which can be packaged and stored in single containers, while avoiding the deleterious consequences of such incompatibilities.

### SUMMARY OF THE INVENTION

The invention is based on the inventors' discovery that drug delivery particles can be made, which particles can contain a “cargo” (e.g. a biologically-active constituent of a medicinal composition). In the context of a composition comprising such particles, the particles can protect the cargo contained therein from potentially deleterious interactions with constituent substances of the composition external to the particle during storage. Further, the particles can ‘release’ said cargo in response to being administered such that the cargo is then free to exert its effect within the body of the recipient subject. In particular, the particles are engineered to be responsive to pH, such that the particle matrix is insoluble at the pH of the final medicinal composition within which the particles are stored, but soluble at the relatively higher pH of the injection site tissue of the subject.

Thus in one aspect, the invention provides a plurality of pH-sensitive drug delivery particles comprising a biologically-active cargo within a matrix, wherein said particles are triggered to release said cargo by being present in an aqueous environment having a higher pH relative to the pH of an aqueous environment in which said particles are present (i.e. stored) prior to being so triggered.

In a further aspect, the invention provides a plurality of pH-sensitive drug delivery particles comprising a biologically-active cargo within a matrix, wherein the amount of cargo released from said plurality of particles when present in an aqueous environment for at least 6 months at a sub-physiological pH is no more than 30 wt % of the total amount of cargo, and wherein on subjecting said particles to a trigger physiological pH (above a threshold pH) the amount of cargo released within 24 hours or less is no less than 50 wt % of the total amount of cargo.

In a further aspect, the invention provides a composition, immunogenic composition or vaccine comprising such a plurality of pH-sensitive drug delivery particles. In a further aspect, the invention provides a vial or parenteral administration device containing such an (immunogenic) composition or vaccine or plurality of pH-sensitive drug delivery particles.

In a further aspect, the invention provides the use in medicine of such a composition, immunogenic composition or vaccine or plurality of pH-sensitive drug delivery particles, in particular for the treatment or prevention of an infection caused directly or indirectly by a pathogen, or of a pathology associated with immunologically distinct host cells such as cancer. In a further aspect, the invention provides a method of eliciting an immune response against an infection- or pathology-causing pathogen or allergen, or immunologically distinct host cells responsible for a pathology such as cancer, comprising the step of administering to a subject an effective amount of such a plurality of particles or composition, immunogenic composition or vaccine.

In a further aspect, the invention provides a method for preventing or reducing interaction between a biologically-active cargo and components of an aqueous environment in which said cargo is present, comprising: forming such a plurality of pH-sensitive drug delivery particles comprising said cargo; and formulating said plurality of particles in said aqueous environment, comprising any necessary adjustment to render said environment of sub-physiological pH.

In a further aspect, the invention provides a method for preventing or reducing interaction between a biologically-active cargo and components of an aqueous environment of sub-physiological pH in which said cargo is present, comprising: forming such a plurality of pH-sensitive drug delivery particles comprising said cargo; and formulating said plurality of particles in said aqueous environment.

In a further aspect, the invention provides a method for making a plurality of drug delivery particles comprising a biologically-active cargo within a matrix, comprising the step of making a solution of said cargo and a matrix polymer and/or the use of a solution comprising a polymer and a cargo. In a further aspect, the invention provides a method for making a plurality of drug delivery particles comprising a biologically-active cargo within a matrix, comprising the steps of: at least partially deprotonating a polymer, which is insoluble in its protonated state, in an aqueous environment such that the polymer has a net negative charge and is soluble in said aqueous environment; combining said polymer with said cargo to produce a stock solution; and forming particles by moulding said stock solution and removing the aqueous environment.

In a further aspect, the invention provides a plurality of drug delivery particles obtainable or obtained by such methods for making a plurality of drug delivery particles.

In a further aspect, the invention provides a method for making a composition, comprising such methods for making a plurality of drug delivery particles and formulating said particles in an aqueous environment. In a further aspect, the invention provides a method for making a composition comprising a plurality of drug delivery particles comprising a biologically-active cargo within a matrix, wherein the matrix comprises a polymer, comprising the steps of: introducing a plurality of particles, made according to such methods for making a plurality of drug delivery particles, to an acidic aqueous environment such that the acidic environment protonates the matrix polymer making the polymer insoluble in said environment; raising the pH of the acidic aqueous environment to a sub-physiological pH which is acceptable for parenteral administration while retaining the insoluble state of the matrix polymer in said environment; and optionally formulating said particles in an aqueous environment of sub-physiological pH.

In a further aspect, the invention provides a composition obtainable or obtained by such methods for making a composition.

### BRIEF DESCRIPTION OF THE DRAWINGS

FIG. 1: Proportion of total Hib (unconjugated and TT-conjugated) in the supernatant fraction as a percentage (wt %) of all Hib present (supernatant+pellet) in the particle-containing sample 700-65 during storage at 4° C., as detected by HPAEC-PAD.

FIG. 2: Proportion of ‘free’ (unconjugated) Hib as a percentage (wt %) of total (i.e. TT-conjugated+free) Hib present in particle-containing sample 700-65 (pellet+supernatant) as detected by HPAEC-PAD during storage at 4° C.

FIG. 3: Proportion of total Hib (unconjugated and TT-conjugated) in the supernatant fraction as a percentage (wt %) of all Hib present (supernatant+pellet) in the particle-containing sample 841-57-1 during storage at 4° C., as detected by HPAEC-PAD.

FIG. 4: Proportion of ‘free’ (unconjugated) Hib as a percentage (wt %) of total (i.e. TT-conjugated+free) Hib present in particle-containing sample 841-57-1 (pellet+supernatant) as detected by HPAEC-PAD during storage at 4° C.

FIG. 5: Total Hib (unconjugated and TT-conjugated) detectable by HPAEC-PAD (in μg/ml) in particle-containing samples 841-57-2 and 841-57-2S (pellet+supernatant) during storage at 4° C.

FIG. 6: Proportion of total Hib (unconjugated and TT-conjugated) in the supernatant fraction as a percentage (wt %) of all Hib present (supernatant+pellet) in aliquots of particle-containing sample 855-118-A respectively stored for approximately four hours at pH 6.5, 6.6, 6.7, 6.8, 6.9, 7.0, 7.2, and 7.4, as detected by HPAEC-PAD.

FIG. 7: Proportion of total Hib (unconjugated and TT-conjugated) in the supernatant fraction as a percentage (wt %) of all Hib present (supernatant+pellet) at various particle concentrations (2 mg/ml, 1 mg/ml and 0.5 mg/ml) of particle-containing sample 855-14 respectively stored for approximately four hours at pH 6.8, 7.4 and 8.0, as detected by HPAEC-PAD.

FIG. 8: (A) Optical microscopy showing dissolution of approximately one year-old sample 700-65 particles, and approximately one week-old sample 817-83-1 particles, as pH is incrementally increased around threshold pH. (B) Optical microscopy showing dissolution of sample 841-12-1 and sample 841-12-3 particles over 10 minutes following sudden increase in sample pH.

FIG. 9: Proportion of ‘free’ (unconjugated) Hib as a percentage (wt %) of total (i.e. TT-conjugated+free) Hib present in particle-containing samples 855-72-2 and 855-72-4 (pellet+supernatant) during storage at 25, 37 and 45° C. (with T=0 value subtracted in each case), as detected by HPAEC-PAD on days 14, 68 and 86. Control sample 855-72-5 contained Hib-TT but no particles.

FIG. 10: Mean anti-Hib antibody titres at 21 (‘PII’) and 35 (‘PIII’) days post-first immunisation of adult rats. The animal groups received, from left to right in FIG. 10: Group 1 (control)—Infanrix Hexa (Infanrix Penta used to extemporaneously reconstitute lyophilized Hib-TT); Group 2 (control)—Infanrix Penta and Hiberix (lyophilized Hib-TT reconstituted in saline) co-administered at different sites; Group 3—Hib-TT-containing particle sample 841-57-1 co-administered at a different site from Infanrix Penta; Group 4—Hib-TT-containing particle sample 841-57-2 mixed with Infanrix Penta and administered after storage at 4° C. for 4 weeks; Group 5—Hib-TT-containing particle sample 700-66 mixed with Infanrix Penta and administered after storage at 4° C. for 16 months.

### ABBREVIATIONS

Hib-CRM: Haemophilus influenzae type b polysaccharide conjugated to diphtheria CRM197 protein; Hib-TT: Haemophilus influenzae type b polysaccharide conjugated to tetanus toxoid; HPAEC-PAD: high pressure anion exchange chromatography with pulsed amperometric detection; kDa: kilodaltons; NaCl: sodium chloride; NaOH: sodium hydroxide; PBS: phosphate buffered saline; PEG: polyethylene glycol; PET: polyethylene terephthalate; PLGA: poly(lactide-co-glycolide) polymer; PMAA: poly(methacrylic acid); PMMA: poly(methyl methacrylate); PMMA-co-PMAA: poly(methyl methacrylate)-co-poly(methacrylic acid) polymer; PRP: polyribosylribitol; PVOH: poly(vinyl alcohol); PVP: poly(vinylpyrrolidone); T: time; THF: tetrahydrofuran; TT: tetanus toxoid; WFI: Water for Injection.

### DETAILED DESCRIPTION OF THE INVENTION

The present invention is concerned with the stable storage and delivery of drug compositions which contain, in a one-part aqueous liquid composition, substances (constituents) which are incompatible such as mutually physically or biochemically reactive or, if the composition is an immunogenic composition or vaccine, prone to interfere immunologically. This is achieved by sequestering at least one of the incompatible substances within micro- or nano-particles in order than it is not exposed to the surrounding environment (i.e. the aqueous composition) containing the substance with which it is incompatible. The substance sequestered within such particles is referred to herein as the “biologically-active cargo”. The particles are engineered to be pH-sensitive, which as referred to herein means being responsive to a pH ‘trigger’ such that below a pre-determined ‘threshold pH’ the cargo remains sequestered within the particles whereas above the threshold pH the cargo is no longer sequestered and is accessible to the surrounding environment. By tuning the particle matrix to have an appropriate threshold pH with respect to the local pH of the target administration site of the intended subject, ‘delivery’ (accessibility of the cargo following ‘release’ from the particles) occurs only after administration.

### Particles

Hence, in one aspect, the invention provides a plurality of pH-sensitive drug delivery particles comprising a biologically-active cargo within a matrix, wherein said particles are triggered to release said cargo by being present in an aqueous environment having a higher pH relative to the pH of an aqueous environment in which said particles are present prior to being so triggered. Put another way, the particles are induced to ‘surrender’ their cargo by an increase, beyond a certain threshold pH, in the pH of the local environment. For example, particles being present as a component of a finally-formulated parenteral composition which maintains a sub-physiological pH level during storage are triggered to release their cargo by the elevated pH level encountered when the composition is injected into the muscle tissue of a human subject.

In some embodiments, the matrix of the particle, within which the biologically-active cargo is comprised, is insoluble in an aqueous environment at a sub-physiological pH, but soluble in an aqueous environment at a ‘trigger’ physiological pH (i.e. at a pH above the threshold pH). As such, in some embodiments, the particles are intact at a sub-physiological pH, whereas on subjecting said particles to a trigger physiological pH said particles are substantially or completely degraded/dissolved within 24 hours or less, for example are at least 80, 85, 90, 95, 99 or 100% degraded/dissolved. The degree to which the particles are intact or degraded/dissolved can be determined in vitro by optical microscopy.

In a further aspect the invention more particularly provides a plurality of pH-sensitive drug delivery particles comprising a biologically-active cargo within a matrix, wherein the amount of cargo released from said plurality of particles when present in an aqueous environment for at least 6 months at a sub-physiological pH is no more than 30 wt % of the total amount of cargo, and wherein on subjecting said particles to a trigger physiological pH (at or above a threshold pH) the amount of cargo released within 24 hours or less is no less than 50 wt % of the total amount of cargo.

By “released” in the context of the above aspect is meant that cargo is detectable in the supernatant, rather than the pellet, of a particle-containing sample after separation of the drug delivery particles e.g. by centrifugation. (Herein, the aqueous part of a particle-containing composition which remains when the particles are separated from it is referred to as the “aqueous environment” or “storage buffer”. This may or may not contain one or more biologically-active constituents.) It is expressed as the proportion of cargo measured as being present in the supernatant relative to the ‘total’ amount detected at the same timepoint, i.e. the supernatant amount plus the amount detected as being associated with the particle-containing fraction e.g. centrifugation pellet. The amount of cargo released can be determined in vitro, such as by HPAEC-PAD. However, it should be noted that more generally the concept of the particles “releasing” cargo as used herein is meant that the cargo is no longer “sequestered” by the particle, in the sense that the cargo is exposed or is accessible to the aqueous environment. Thus, except as mentioned above in the specific context of quantifying association of cargo with the particles, “released” is not intended necessarily to imply a physical dissociation or separation of the cargo from the particle matrix; rather it means that the particle structure or integrity has been altered by the change in pH in such a way that cargo becomes accessible to the local aqueous environment.

Reference to the particles “comprising a biologically-active cargo within a matrix” as used herein is intended to encompass various ways in which such a cargo and a matrix material can together form a particle. When ‘comprised within a matrix’ in this sense, the cargo can be said to be sequestered, meaning it is largely or entirely inaccessible to the external environment, e.g. the aqueous environment of the composition. In some embodiments, the cargo is encapsulated within the matrix. In preferred embodiments, the cargo is substantially homogeneously dispersed throughout or entangled with the matrix of the particle.

In some embodiments, the aqueous environment at sub-physiological pH comprises a buffer, such as a saline, phosphate, Tris, borate, succinate, histidine, citrate or maleate buffer.

As used herein, the meaning of “sub-physiological pH” and “physiological pH” is with respect to the local physiology of the intended recipient subject of the particles (as formulated into an administrable composition), i.e. the pH of the tissue of the subject's injection site. In preferred embodiments, “sub-physiological pH” and “physiological pH” respectively mean sub-physiological and physiological with respect to the pH of human tissue, in particular human muscle and/or infant tissue. In some embodiments, the sub-physiological pH differs from the physiological pH by at least 0.1, 0.2, 0.3, 0.4, 0.5, 0.6, 0.7, 0.8, 0.9, 1.0, 1.1, 1.2, 1.3, 1.4 or 1.5 pH units, i.e. the physiological pH is at least 0.1, 0.2, 0.3, 0.4, 0.5, 0.6, 0.7, 0.8, 0.9 or 1.0, 1.1, 1.2, 1.3, 1.4 or 1.5 pH units higher than the sub-physiological pH. These values may represent the lower limit of a range which is bounded at the upper end by a value selected from 0.2, 0.3, 0.4, 0.5, 0.6, 0.7, 0.8, 0.9, 1.0, 1.1, 1.2, 1.3, 1.4, 1.5, 2.0 or 3.0. In some embodiments, the sub-physiological pH is at or below 6.8, 6.7, 6.6, 6.5, 6.4, 6.3, 6.2, 6.1 or 6.0, or comprises a range with these respective values as the upper limit and a lower limit selected from 6.7, 6.6, 6.5, 6.4, 6.3, 6.2, 6.1, 6.0, 5.9, 5.8, 5.7, 5.6, 5.5, 5.4, 5.3, 5.2, 5.1 or 5.0. In some embodiments, the physiological pH is at or above 6.8, 6.9, 7.0, 7.1, 7.2, 7.3, 7.4, 7.5, 7.6 or 7.7, or comprises a range with these respective values as the lower limit and an upper limit selected from 6.9, 7.0, 7.1, 7.2, 7.3, 7.4, 7.5, 7.6, 7.7, 7.8, 7.9, 8.0, 8.1, 8.2, 8.3, 8.4 or 8.5.

In some embodiments, the amount of cargo released from said plurality of particles when present in an aqueous environment for at least 6 months at a sub-physiological pH is less than or equal to 30 wt % of the total amount of cargo, such as no more than 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 wt %. These values may represent the upper limit of a range which is bounded at the lower end by a value selected from 29, 28, 27, 26, 25, 24, 23, 22, 21, 20, 19, 18, 17, 16, 15, 14, 13, 12, 11, 10, 9, 8, 7, 6, 5, 4, 3, 2 or 1. Such a level of release of cargo (conversely, such a level of sequestration within the particles) when present in an aqueous environment at a sub-physiological pH is, in some embodiments, achievable over a longer duration, such as at least 7, 8, 9, 10, 11, 12, 14, 16, 18, 20, 22, 24, 26, 28, 30, 32, 34 or 36 months. These values may represent the lower limit of a range which is bounded at the upper end by a value selected from 8, 9, 10, 11, 12, 14, 16, 18, 20, 22, 24, 26, 28, 30, 32, 34, 36, 48 or 60 months.

The amount of cargo released from said particles within 24 hours or less of subjecting said particles to a trigger physiological pH is, in some embodiments, greater than or equal to 50 wt % of the total amount of cargo, such as no less than 50, 55, 60, 65, 70, 75, 80, 85, 90, 95, 96, 97, 98, 99 or 100 wt %. These values may represent the lower limit of a range which is bounded at the upper end by a value selected from 55, 60, 65, 70, 75, 80, 85, 90, 95, 96, 97, 98, 99 or 100. Such a level of release of cargo in response to a trigger physiological pH is, in some embodiments, achievable over a shorter duration, such as within 20, 16, 12, 10, 8, 6, 4, 2 or 1 hours or 45, 30, 15, 10 or 5 minutes. These values may represent the upper limit of a range which is bounded at the lower end by a value selected from 16, 12, 10, 8, 6, 4, 2 or 1 hours or 45, 30, 15, 10, 5 or 1 minutes.

In some embodiments, the sub-physiological pH aqueous environment in which the particles are present for at least 6 months is maintained at between 2-8° C., e.g. at about 4° C. However, in some embodiments, the particles of the invention are thermostable. This means that while present in the sub-physiological pH aqueous environment at a temperature in the range 2-8° C. (i.e. not exceeding 2, 3, 5, 6, 7, 8 or, preferably, 4° C.), the particles may be subjected to a temperature excursion (i.e. a temperature exceeding 2, 3, 4, 5, 6, 7 or 8° C.) not exceeding about 25° C. or 37° C. for up to 12 weeks, such as for a duration of between 1 day and 2, 4, 6, 8, 10 12 weeks.

In some embodiments, subjecting the particles to a trigger physiological pH occurs at a temperature of around the body temperature of the recipient, in particular at around the temperature of injection site tissue of the recipient, such as at or around 37° C. in the case of a human.

In some embodiments, the particles are highly uniform with respect to shape, size and/or composition, for example as a result of being moulded. One way in which such particles may be fabricated is using PRINT™ Technology (Liquidia Technologies, Inc.), which is a method capable of forming (micro- and/or nano-) particles that: (i) are monodisperse in size and uniform shape; (ii) can be moulded into any shape; (iii) can be comprised of essentially any matrix material; (iv) can be formed under mild conditions (compatible with delicate cargoes); (v) are amenable to post-functionalisation chemistry (e.g. bioconjugation of active agents and/or targeting components); and (vi) which initially fabricates particles in an addressable 2D array (which opens up combinatorial approaches since the particles can be “bar-coded”). The methods and materials for fabricating the particles of the present invention are further described and disclosed in the co-applicant's issued patents and co-pending patent applications, each of which are incorporated herein by reference in their entirety: U.S. Pat. Nos. 8,518,316; 8,444,907; 8,420,124; 8,268,446; 8,263,129; 8,158,728; 8,128,393; 7,976,759; U.S. Pat. Application Publications Nos. 2013-209564; 2013-0249138, 2013-0241107, 2013-0228950, 2013-0202729, 2013-0011618, 2013-0256354, 2012-0189728, 2011-151015, 2010-0003291, 2009-0165320, 2008-0131692; PCT Publication No. WO2015/073831; and pending application Ser. No. 13/852,683 filed Mar. 28, 2013 and Ser. No. 13/950,447 filed Jul. 25, 2013.

Particles produced using PRINT™ Technology are made by moulding the materials intended to make the particles in mould cavities. The PRINT™ Technology generally utilizes low surface energy moulds made from materials such as silicones, perfluoro-polyether-based elastomers (PFPEs) or other hydrocarbon-based materials to replicate micro or nano sized structures on a master template. The polymers utilized in moulds are often liquids at room temperature and may be photo-chemically cross-linked into elastomeric solids that enable high resolution replication of micro- or nano-sized structures. The liquid polymer is ‘solidified’ while in contact with a master template, thereby forming a replica image of the structures on the master template. Solidification of the mould in contact with the master template can take place by curing (thermally or photochemically), by cooling down by vitrification, and/or by crystallization. Upon removal of the polymer mould from the master template, the polymer forms a patterned template that includes cavities or recess replicas of the micro or nano-sized features of the master template. The micro or nano-sized cavities in the patterned template can be used for high-resolution particle fabrication. PRINT™ Technology enables the fabrication of monodisperse organic and inorganic particles with simultaneous control over structure (e.g., shape, size and composition) and function (e.g., surface structure). The monodisperse nature of the particles in terms of physical and compositional make-up provides for highly uniform and pre-determinable particle properties such as rates of particle degradation/dissolution and thereby cargo release rates and dosing ranges.

Technical aspects to be considered when designing a particle carrier system using PRINT′″ Technology include, among others: (i) compatibility of the particle cargo or matrix materials with the polymer PRINT™ mould materials; (ii) particle degradation/dissolution profile desired for cargo release, (iii) surface functionalization for particle targeting or particle compatibility, (iv) particle modulus; and (v) the combination of points (i)-(iv) in the formation of a particle precursor solution that is amenable to the moulding process. Cargo compatibility within a particle matrix can be addressed, for example, by tuning the hydrophilicity of the matrix to match that of the cargo through judicious choice of matrix materials. Particle degradation/dissolution is discussed herein. Modulus of the particles can be adjusted by changing, for example, the constituents of the particle. Finally, the particle precursor can be optimized for particle fabrication, if needed, by adding co-monomers or co-solvents to alter the physical properties of the particle precursor solution.

In embodiments of the invention in which the particles are moulded, the particles thereby produced will have a size and shape that substantially mimics the size and shape of the cavity of the mould in which each particle was formed. Depending on the dimension of a mould cavity, the particles may be microparticles or nanoparticles. By selecting a mould of appropriate dimensions, the size and shape of the particles can be tuned to meet specific delivery needs such as e.g. cargo loading, degradation/dissolution rate, etc. In some embodiments, microparticles according to the present invention can have a largest dimension of less than about 1000 μm, less than about 900 μm, less than about 800 μm, less than about 700 μm, less than about 600 μm, less than about 500 μm, less than about 400 μm, less than about 300 μm, less than about 200 μm, less than about 100 μm, less than about 50 μm, less than about 10 μm, less than about 5 μm, or about 1 μm. In other embodiments, the particles are nanoparticles and can have a largest dimension of less than about 1000 nm, less than about 900 nm, less than about 800 nm, less than about 700 nm, less than about 600 nm, less than about 500 nm, less than about 400 nm, less than about 300 nm, less than about 200 nm, less than about 100 nm, or less than about 50 nm. It will be appreciated by a person having ordinary skill in the art that mould cavities and corresponding particles produced from those moulds can have a dimension falling between the sizes explicitly mentioned above. Moreover, the dimension may be a length, width, or diameter of the particle.

In some embodiments, the longest axis of the particles is between about 1-10 μm, in particular between 5-7 μm, such as 6 μm. In some embodiments, the particles have at least one axis which is less than 200 nm, and optionally may be sterile filterable.

It will also be appreciated by a person having ordinary skill in the art that particles may be dimensioned to have selected aspect ratios. As defined herein, “aspect ratio” describes the ratio of the longest axis to the shortest axis of a particle. In some embodiments, the aspect ratio is at least 1:1, at least 2:1, at least 5:1, at least 10:1, at least 50:1, or at least 100:1. In particular embodiments, the aspect ratio is between about 1:1 and about 5:1, between about 5:1 and about 10:1, or between about 10:1 to 100:1. Particles may be moulded into any desired shape. In some embodiments, the particles are donut-shaped or rod-shaped. In some embodiments, the particles are for parenteral administration, such as intradermal or subcutaneous or, preferably, intramuscular administration.

### Particle Matrix

The particles of the present invention comprise a biologically-active cargo within a matrix. The matrix provides a structural substrate for forming particles and influences particle stability and the kinetics of their degradation/dissolution in response to a pH trigger. The particles of the invention therefore have tunable cargo release profiles, in part through the selection of matrix materials and their relative proportions, etc. The matrix of the particles may be manufactured using a variety of materials including synthetic proteins, natural proteins, recombinant proteins, peptides, synthetic polymers, bioabsorbable polymers, polysaccharides, nucleic acids, small molecules, or any combination thereof. Suitable bioabsorbable polymers include poly(vinyl alcohol) (PVOH), polyethylene glycol (PEG), polyacrylic acid, polyacrylamide, poly(vinylpyrrolidone) (PVP), synthetic or natural polyamino acids, and PMMA-co-PMAA. Suitable polysaccharides include dextran, dextran derivatives, chitosan, chitosan derivatives, hyaluronic acid, alginic acid, agarose, pectin, cellulosics, cellulosic derivatives, cellulose ethers, xanthan gum, carrageenan, guar gum, starch, and inulin. Gelatin is also suitable for the matrix of the particle.

Thus in some embodiments, the matrix is polymeric, i.e. is comprised of one or more polymers. The polymer may be a homopolymer, or a hetero- or co-polymer, such as an alternating or block copolymer. Preferably, such polymeric matrix is biocompatible, biodegradable, bioresorbable and/or excretable in or from the human body. In some embodiments the polymer of the polymeric matrix, when in the aqueous environment at sub-physiological pH, is in an at least partially protonated state and/or may have an approximately or exactly neutral charge and be insoluble.

In some embodiments, the polymeric matrix comprises a polymer having a pKa below the threshold physiological pH.

In some embodiments, the polymeric matrix comprises (poly(methyl methacrylate)-co-poly(methacrylic acid) copolymer (PMMA-co-PMAA copolymer); poly(glutamic acid)-co-poly(lysine); zwitterionic hetero- or homo-poly(amino acids); carboxymethyl chitosan; hypromellose phthalate; hypromellose acetate succinate; or an acrylate co-polymer represented by the general formula (1) wherein:

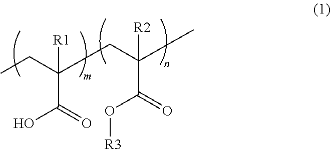

R1 represents hydrogen or methyl, R2 represents hydrogen or methyl, and R3 represents methyl, ethyl, propyl, isopropyl, n-butyl, iso-butyl, tert-butyl, sec-butyl, phenyl, or benzyl. The PMMA-co-PMAA copolymer may have a weight average molecular weight (Mw) in the range 1-200 kDa, such as in the range 50-60 kDa or 35-45 kDa or 22-28 kDa or 8-12 kDa, such as a weight average molecular weight (Mw) of 10, 25, 40, 55 or 125 kDa.

In some embodiments, the polymeric matrix comprises PMMA-co-PMAA copolymer, wherein the molar ratio of methyl methacrylate (MMA) monomer to methacrylic acid (MAA) monomer in the copolymer is in the range 1:1-4:1, such as in the range 1.5-2:1. In other embodiments, the polymeric matrix comprises poly(glutamic acid)-co-poly(lysine), wherein the molar ratio of glutamic acid monomer to lysine monomer is approximately or exactly 1:1.

### Cargoes

The pH-sensitive drug delivery particles of the present invention comprise, in addition to the above-discussed matrix, a biologically-active cargo. Such cargo is sequestered within the particles, permitting storage within an aqueous environment while preventing any undesirable interactions between the cargo and components of the aqueous environment. By “biologically-active” as used herein in connection with the cargo is meant that the cargo is not inert with respect to the biological (e.g. physiological, immunological, etc) functioning of the body of the recipient to which the particles are administered. In other words, such biologically-active cargo is capable of interacting with the body of the recipient to mediate some manner of biological effect. The biological effect may be a therapeutic or prophylactic effect, and the cargo may therefore be a “drug”, which in connection with the particles of the invention is herein is used in its broadest sense. Therefore the cargo may be an active agent, a pharmaceutical agent, a therapeutic agent, or a vaccine agent. In some embodiments, the particles may each comprise more than one biologically-active cargo, such as one, two, three or four or more different cargoes. In some embodiments, the particles may each comprise more than one biologically-active cargo that are of the same type, such as two, three, four, or more drugs or two, three, four, or more therapeutic agents. In some embodiments, the particles may each comprise more than one biologically-active cargo that are of different types, such as one, two, three, four or more drugs and one, two, three, or four or more vaccine agents.

The biologically-active cargo may more particularly comprise an antigen, antibody, small-molecule drug compound, immunoglobulin, protein, polysaccharide, protein-polysaccharide conjugate, nucleic acid or adjuvant (non-specific immunomodulatory agent). The biologically-active cargo may in some embodiments be hydrolytically-sensitive meaning that, subject to prevailing parameters such as pH, temperature, ionic strength etc, the cargo is susceptible to a material degree of hydrolytic degradation when in contact with an aqueous environment. For example, a “material” degree of hydrolytic degradation might be, in the context of an antigen cargo, a degree of degradation which causes a detectable reduction in immunogenicity or antigenicity. In some embodiments, the biologically-active cargo has a low isoelectric point (pI), such as a pI of 4 or below, in particular 3 or 2 or below. In some embodiments, the biologically-active cargo comprises phosphate groups, such as in phosphodiester bonds.

In particular embodiments, the biologically-active cargo comprises an antigen. The term “antigen” is well-understood by those of skill in the art to mean an agent capable of eliciting an immune response in a human or animal body. Antigens are therefore the ‘active ingredients’ in immunogenic compositions/vaccines. An antigen may comprise or consist of, for example, a protein or polypeptide, a saccharide such as an oligo- or poly-saccharide, a conjugate of a protein and a saccharide or a nucleic acid. Antigens may be presented in various forms, such as purified or recombinant proteins, polysaccharides, conjugates of such proteins and polysaccharides, nucleic acid vectors for in vivo antigen production, inactivated whole bacteria or viruses, viral fragments, virus-like particles, live attenuated bacteria, replicating attenuated viruses or bacterial outer membrane complexes. Antigens, being cargo according to the invention, can be any type of antigen as described above, and may be antigens derived from or related to a pathogen (such as a bacteria, virus or other pathogen), a cancer/tumour, an allergic or autoimmune condition, a non-infectious disease condition, an addiction condition, or any other physiological condition potentially amenable to prophylatic or therapeutic intervention via immunisation.

In some embodiments wherein the biologically-active cargo is an antigen, said cargo comprises a saccharide such as an oligo- or polysaccharide. The expression “oligo/polysaccharide” will be used herein to mean an oligosaccharide or polysaccharide which has been isolated from a pathogen. In some such embodiments, the oligo/polysaccharide has a low isoelectric point (pI), such as a pI of 4 or below, in particular 3 or 2 or below. The oligo/polysaccharide may be used in its native form as isolated from the pathogen, or may be processed. Such processing may be, e.g. sizing of the native saccharides by e.g. microfluidisation (other techniques are described in EP0497524).

In some embodiments, the oligo/polysaccharide is derived from a bacterial pathogen and in particular may be derived from bacterial capsular saccharide or lipooligosaccharide (LOS) or lipopolysaccharide (LPS). For example, the oligo/polysaccharide may be derived from a bacterial pathogen selected from the group consisting of: Haemophilus influenzae type b (“Hib”); Neisseria meningitidis (in particular serotypes A, C, W and/or Y); Streptococcus pneumoniae (in particular serotypes 1, 2, 3, 4, 5, 6A, 6B, 7F, 8, 9N, 9V, 10A, 11A, 12F, 14, 15B, 15C, 17F, 18C, 19A, 19F, 20, 22F, 23F and/or 33F); Staphylococcus aureus, Bordetella pertussis, and Salmonella typhi.

In a particular embodiment, the saccharide in said saccharide-comprising antigen cargo is an oligo/polysaccharide conjugated to a carrier protein, i.e. said cargo is an oligo/polysaccharide-protein conjugate antigen. Such conjugates are well-known in the art as a means to confer upon the oligo/polysaccharide antigen the T-cell dependent character of the immune response elicited by the carrier protein. Hence, carrier proteins are selected for their ability to provide a source of T-helper cell epitopes. In a given oligo/polysaccharide-protein conjugate, the carrier protein may be derived from the same pathogen as the oligo/polysaccharide, or from a different pathogen. Carrier proteins suitable for use in the oligo/polysaccharide-protein conjugate antigen cargoes of the invention are well known in the art, and include: tetanus toxoid, fragment C of tetanus toxoid, diphtheria toxoid, CRM197 or another non-toxic mutant of diphtheria toxin, protein D of non-typeable Haemophilus influenzae, outer membrane protein complex (OMPC) of Neisseria meningitidis, pneumococcal PhtD, pneumococcal pneumolysin, exotoxin A of Pseudomonas aeruginosa (EPA), detoxified haemolysin of Staphylococcus aureus, detoxified adenylate cyclase of Bordetella sp, detoxified Escherichia coli heat labile enterotoxin, or cholera toxin subunit B (CTB) or detoxified cholera toxin.

As discussed above, the biologically-active cargo of the particles of the invention may be hydrolytically sensitive. In the case of an oligo/polysaccharide-protein conjugate antigen, hydrolytic sensitivity can manifest as hydrolytic cleavage within the saccharide chain or between the saccharide and carrier protein, in either case resulting in the production of ‘free’ (unconjugated) saccharide, i.e. saccharide that is not conjugated to protein, which is not desirable. As the sequestration of the oligo/polysaccharide-protein conjugate antigen within the particles of the invention serves to protect the conjugate antigen from possible hydrolytic interactions with a (sub-physiological pH) aqueous environment in which the particles may be present, loss of conjugate integrity during storage of the particles in an aqueous environment is minimised. Thus in some embodiments wherein the biologically-active cargo is an oligo/polysaccharide-protein conjugate antigen, the amount of free (unconjugated) saccharide, derived from said oligo/polysaccharide conjugate, present collectively in the drug delivery particles and aqueous environment is no more than 30 or 25 or 20 or 15 or 10 wt % of the total amount of conjugated and free saccharide present collectively in the particles and aqueous environment during the at least 6 months in an aqueous environment at sub-physiological pH. These values may respectively represent the upper end of a range which is bounded at the lower end by a value selected from 25, 20, 15, 10 or 5 wt %. In some such embodiments, such a maximum level of free saccharide increase applies during a period of longer than 6 months, such as at least 7, 8, 9, 10, 11, 12, 14, 16, 18, 20, 22, 24, 26, 28, 30, 32, 34 or 36 months. These values may represent the lower limit of a range which is bounded at the upper end by a value selected from 8, 9, 10, 11, 12, 14, 16, 18, 20, 22, 24, 26, 28, 30, 32, 34, 36, 48 or 60 months. In some such embodiments, the sub-physiological pH aqueous environment in which the particles are present for at least 6 months is maintained at between 2-8° C. In particular embodiments, the particles may be subjected to a single excursion to a temperature exceeding this range, however not exceeding about 37° C. for no longer than about 2 weeks. Preferably, the excursion does not exceed about 25° C.

In preferred embodiments, the biologically-active cargo comprises oligo/polysaccharide derived from the capsular saccharide of Haemophilus influenzae type b (“Hib”; polyribosylribitol phosphate or “PRP”), optionally in its full-length native form, conjugated to CRM197 or, more preferably, tetanus toxoid. In other preferred embodiments, the oligo/polysaccharide is derived from capsular saccharide from Neisseria meningitidis, in particular serotype A. In these preferred embodiments, the conjugate antigens are preferably substantially homogeneously dispersed throughout the matrix of the particles.

### Compositions

The pH-sensitive drug delivery particles of the invention may be stored and delivered to a subject, being an animal, in particular a mammal, more particularly a human, in a parenterally-acceptable composition. Hence, in one aspect of the invention is provided a composition comprising a plurality of pH-sensitive drug delivery particles of the invention in an aqueous, preferably sterile, environment. Such a composition of the invention may be an immunogenic composition, i.e. a composition capable of eliciting in a subject an immune response directed specifically to one or more antigenic components present in the composition. Such an immunogenic composition may be a vaccine. Put another way, the invention provides a vaccine comprising a (immunogenic) composition of the invention as described herein.

Preferably such compositions maintain a pH below the threshold physiological pH of the particles or, put another way, are of sub-physiological pH. Such sub-physiological pH of the composition or the aqueous environment thereof is determined relative to the local physiological pH of the particular tissue type/anatomical region (e.g. intramuscular, intravenous) of the particular subject type (e.g. animal, mammal, human, adult, infant) to which the composition is intended to be directly administered. In some embodiments, the composition/aqueous environment is adjusted to have a pH at or below 6.8, 6.7, 6.6, 6.5, 6.4, 6.3, 6.2, 6.1 or 6.0. The values may respectively define the upper end of a range which is defined at the lower end by a value selected from 6.7, 6.6, 6.5, 6.4, 6.3, 6.2, 6.1, 6.0, 5.9, 5.8, 5.7, 5.6, 5.5, 5.4, 5.3, 5.2, 5.1 or 5.0. The aqueous environment may contain one or more physiologically acceptable excipients and/or a buffer such as a saline, phosphate, Tris, borate, succinate, histidine, citrate or maleate buffer.

The composition may comprise more than one population of pH-sensitive particles, each population containing different cargoes and comprising the same or different particle matrices. Thus, in some embodiments the composition comprises a first plurality and a second plurality of particles, wherein said second plurality of particles comprises a cargo other than the cargo of the first plurality of particles. Alternatively, the composition may contain a plurality of populations of particles differing in physical characteristics such as matrix polymer, size, and shape; such populations may respectively comprise the same or different cargoes.

In some embodiments, for example wherein the polymeric matrix of the particles comprises PMMA-co-PMAA copolymer, the particles are present in the composition at a concentration of 0.1-15, 0.5-12.5, 1-10 or 2-5 mg/ml, such as 0.5-3 mg/ml, in particular 1.0-2.5 mg/ml. In such embodiments the PMMA-co-PMAA copolymer may, for example, have a molar ratio of methyl methacrylate (MMA) monomer to methacrylic acid (MAA) monomer in the range 1:1-4:1 (such as in the range 1.5-2:1) and may have a weight average molecular weight (Mw) in the range 1-200 kDa, such as in the range 50-60 kDa or 35-45 kDa or 22-28 kDa or 8-12 kDa, such as a weight average molecular weight (Mw) of 8.5, 10, 23.9, 25, 37, 40, 51, 55 or 125 kDa.

As a result of the cargo being sequestered within the matrix of the particles, accessibility of the cargo to the aqueous environment is impaired or substantially prevented. The cargo is therefore substantially prevented by the matrix from interacting with components of the aqueous environment, or such interaction is at least reduced relative to the situation in the absence of the particle matrix. In some embodiments, said interaction is prevented or reduced for at least 6 months, such as 7, 8, 9, 10, 11, 12, 14, 16, 18, 20, 22, 24, 26, 28, 30, 32, 34 or 36 months (these values may represent the lower limit of a range which is bounded at the upper end by a value selected from 8, 9, 10, 11, 12, 14, 16, 18, 20, 22, 24, 26, 28, 30, 32, 34, 36, 48 or 60 months); in some such embodiments, the composition is maintained at about 4° C.

In some embodiments, wherein the composition is an immunogenic composition, the aqueous environment (i.e. not including the particles present therein) comprises one or more antigens, and optionally associated components such as one or more adjuvants. Such an adjuvant may have a high pI, such as a pI of 8 or above, such as 9 or 10 or above, in particular 11 or above. In a preferred embodiment the adjuvant is aluminium hydroxide.

The one or more antigens comprised in the aqueous environment in some embodiments of the immunogenic composition may, in some embodiments, be selected from: diphtheria toxoid, tetanus toxoid, acellular pertussis antigens (such as pertussis toxoid, filamentous haemagglutinin, pertactin), Hepatitis B Surface Antigen (HBsAg) and Inactivated Polio Vaccine (IPV), Haemophilus influenzae type b oligo/polysaccharide conjugate antigen, N. meningitidis serotype C oligo/polysaccharide conjugate antigen, N. meningitidis serotype A oligo/polysaccharide conjugate antigen, N. meningitidis serotype W oligo/polysaccharide conjugate antigen, N. meningitidis serotype Y oligo/polysaccharide conjugate antigen and N. meningitidis serotype B antigen. In particular, the following combinations of antigens may be comprised within the aqueous environment of immunogenic compositions of the invention:
i. diphtheria toxoid and tetanus toxoid;
ii. diphtheria toxoid, tetanus toxoid, acellular pertussis antigens;
iii. diphtheria toxoid, tetanus toxoid, acellular pertussis antigens and HBsAg;
iv. diphtheria toxoid, tetanus toxoid, acellular pertussis antigens and IPV;
v. diphtheria toxoid, tetanus toxoid, acellular pertussis antigens, HBsAg and IPV;
vi. Neisseria meningitidis serotype C (MenC) capsular oligo/polysaccharide conjugated to carrier protein, Neisseria meningitidis serotype W (MenW) capsular oligo/polysaccharide conjugated to carrier protein and Neisseria meningitidis serotype Y (MenY) capsular oligo/polysaccharide conjugated to carrier protein;
vii. Neisseria meningitidis serotype C (MenC) capsular oligo/polysaccharide conjugated to carrier protein, Neisseria meningitidis serotype W (MenW) capsular oligo/polysaccharide conjugated to carrier protein, Neisseria meningitidis serotype Y (MenY) capsular oligo/polysaccharide conjugated to carrier protein and antigen derived from Neisseria meningitidis serotype B (MenB).

Preferably, wherein the aqueous environment of the immunogenic composition comprises one of combinations (i)-(v) above, the cargo is a Hib oligo/polysaccharide conjugate antigen. Also preferably, wherein the aqueous environment of the immunogenic composition comprises one of combinations (vi)-(vii) above, the cargo is a MenA oligo/polysaccharide conjugate antigen. In some such embodiments wherein diphtheria toxoid, tetanus toxoid, acellular pertussis antigens and/or HBsAg are present in the aqueous environment, the diphtheria toxoid, tetanus toxoid, acellular pertussis antigens are adsorbed onto aluminium hydroxide and the HBsAg is adsorbed onto aluminium phosphate. In some such embodiments, diphtheria toxoid is present at the amount per dose of 1-10 International Units (IU) (for example exactly or approximately 2 IU) or 10-40 IU (for example exactly or approximately 20 or 30 IU) or 1-10 Limit of flocculation (Lf) units (for example exactly or approximately 2 or 2.5 or 9Lf) or 10-30Lf (for example exactly or approximately 15 or 25Lf), and tetanus toxoid is present at the amount per dose of 10-30 IU (for example exactly or approximately 20 IU) or 30-50 IU (for example exactly or approximately 40 IU) or 1-15Lf (for example exactly or approximately 5 or 10Lf).

In some embodiments, in addition to particle-associated Hib oligo/polysaccharide conjugate antigen, the immunogenic composition comprises, in its aqueous environment, diphtheria toxoid and tetanus toxoid at the respective exact or approximate amounts per dose: 30:40 IU; 25:10Lf; 20:40 IU; 15:5Lf; 2:20 IU; 2.5:5Lf; 2:5Lf; 25:10Lf; 9:5Lf. Acellular pertussis (Pa) antigens including pertussis toxoid (PT), filamentous haemagglutinin (FHA) and pertactin (PRN) may also be present, such that the aqueous environment comprises DTPa antigens in the following amounts:
20-30 ug, for example exactly or approximately 25 ug of PT;
20-30 ug, for example exactly or approximately 25 ug of FHA;
1-10 ug, for example exactly or approximately 3 or 8 ug of PRN;
10-30Lf, for example exactly or approximately 15 or 25Lf of D; and
1-15Lf, for example exactly or approximately 5 of 10Lf of T; or
2-10 ug, for example exactly or approximately 2.5 or 8 ug of PT;
2-10 ug, for example exactly or approximately 5 or 8 ug of FHA;
0.5-4 ug, for example 2-3 ug such as exactly or approximately 2.5 or 3 ug of PRN;
1-10Lf, for example exactly or approximately 2 or 2.5 or 9Lf of D; and
1-15Lf, for example exactly or approximately 5 of 10Lf of T.

As mentioned above, interaction between the cargo and components of the aqueous environment is reduced or substantially prevented by the particle matrix. In this way, in embodiments of the immunogenic compositions of the invention, the particle matrix prevents or reduces aggregation or flocculation and/or prevents or reduces immunological interference and/or prevents hydrolytic degradation of the cargo, relative to an equivalent composition wherein the cargo is not sequestered within particles and is accessible to the aqueous environment. In some embodiments, said interaction, and in particular said aggregation/flocculation and/or immunological interference and/or hydrolytic degradation, is prevented or reduced for at least 6 months, such as 7, 8, 9, 10, 11, 12, 14, 16, 18, 20, 22, 24, 26, 28, 30, 32, 34 or 36 months (these values may represent the lower limit of a range which is bounded at the upper end by a value selected from 8, 9, 10, 11, 12, 14, 16, 18, 20, 22, 24, 26, 28, 30, 32, 34, 36, 48 or 60 months), optionally wherein said composition is maintained at about 4° C.

The phenomenon of aggregation or flocculation, which may be observed visually or by optical microscopy, occurs when certain cargoes interact with certain components of the aqueous environment, resulting in the formation of a network of particles. In the case of an immunogenic composition containing an antigen cargo, such a network of particles may mask epitopes and ‘interfere’ negatively with the elicited immune response. In some cases, the aggregation/flocculation and resulting interference may be the result of the cargo and aqueous environment component having respectively low and high (or vice versa) isoelectric points (pI), such that they are drawn to interact with each other. This is thought to be the reason for observed aggregation/flocculation/interference between the PRP saccharide of Hib conjugate vaccine (low pI) and the aluminium hydroxide adjuvant (high pI) used to adsorb other antigens in some Hib conjugate-containing combination vaccines. Thus, in some embodiments, said cargo has a low pI such as a pI of 4 or below, in particular 3 or 2 or below and/or the aqueous environment comprises a component having a high pI, such as a pI of 8 or above, such as 9 or 10 or 11. Such low pI cargo may comprise phosphate groups, e.g. in the context of phosphodiester bonds. However, the immunological interference reduced or prevented by the immunogenic compositions provided herein is not necessarily associated with flocculation or aggregation. In embodiments wherein the aqueous environment comprises one or more antigens, preferably the particle matrix does not interfere with the immunogenicity of said one or more antigens.

In particular embodiments of the immunogenic compositions of the invention, the matrix of the particles prevents or reduces aggregation or flocculation of the Hib-TT or Hib-CRM197 or MenA-CRM197 and/or prevents or reduces immunological interference on the Hib-TT or Hib-CRM197 or MenA-CRM197.

Hydrolytic degradation, such as cleavage, of hydrolytically-sensitive cargoes is discussed above, and can occur through interaction of said cargo with water molecules in the aqueous composition. Therefore, preventing or minimising exposure of the cargo to the water molecules can reduce or prevent the occurrence of hydrolysis. Accordingly, the present immunogenic compositions achieve this through sequestration of the antigen cargo within the particle. Some saccharide-based antigens, such as Hib and MenA, can be particularly prone to hydrolytic degradation, such as depolymerisation. In particular embodiments of the immunogenic compositions of the invention, the matrix of the particles prevents or reduces hydrolytic degradation of the Hib-TT or Hib-CRM197 or MenA-CRM197.

The immunogenic compositions of the invention, in some embodiments, are suitable for parenteral administration. The person skilled in the art is aware of how to formulate therapeutic compositions for compatibility with a given parenteral route of administration, e.g. intramuscular. In particular, the skilled person knows how to formulate such compositions to be of a particular pH, this being a key characteristic of the immunogenic compositions of at least some embodiments of the invention wherein the aqueous environment in which the particles are administered (and optionally stored) is of sub-physiological pH.

The invention further provides a plurality of particles or composition of the invention, packaged in a therapeutically-suitable container. The particles or composition may be presented in a vial from which the contents may be extracted when needed, for example using a needle and syringe. Alternatively particles or composition may be pre-filled in a parenteral administration device such as a syringe. Such a syringe may be a conventional single-chambered syringe, or may be a dual-chambered syringe. The dual-chambered syringe may be designed to deliver the respective contents of the chambers sequentially, or simultaneously following extemporaneous mixing within the syringe.

### Use and Administration of Drug Delivery Particles

In an aspect of the invention is provided a method for preventing or reducing interaction between a biologically-active cargo and components of an aqueous environment in which said cargo is present, comprising (i) forming a plurality of pH-sensitive drug delivery particles as defined herein comprising said cargo, and (ii) formulating said plurality of particles in said aqueous environment, comprising any necessary adjustment to render said environment of sub-physiological pH. (In step (ii) above, said adjustment may not be necessary if the aqueous environment is already at sub-physiological pH.) Thus, in one embodiment of this aspect of the invention is provided a method for preventing or reducing interaction between a biologically-active cargo and components of an aqueous environment of sub-physiological pH in which said cargo is present, comprising (i) forming a plurality of pH-sensitive drug delivery particles as defined herein comprising said cargo, and (ii) formulating said plurality of particles in said aqueous environment. In some embodiments, the result of formulating according to the steps (ii) above is the production of a composition, immunogenic composition, or vaccine as defined herein.

As discussed above, such prevention or reduction of said interaction is advantageous in various circumstances. Accordingly, in some embodiments, the above methods are methods for storing a biologically-active cargo in an aqueous environment (wherein the storage is effected by the prevention or reduction of interactions between the biologically-active cargo and components of an aqueous environment). Similarly, in some such embodiments as well as in other embodiments, the above methods are for preventing or reducing interaction between said biologically-active cargo and water molecules in said aqueous environment, or between said biologically-active cargo and a component of the aqueous environment other than water. Such a component may be, for example, an adjuvant or antigen or biological or pharmaceutical active ingredient, or a formulation excipient. In particular, said methods may be for preventing or reducing degradation, such as hydrolytic degradation (i.e. through interaction with water molecules), of said cargo in said aqueous environment.

Wherein the above methods are for storing a biologically-active cargo in an aqueous environment, said storing may be for at least 6 months, for example 7, 8, 9, 10, 11, 12, 14, 16, 18, 20, 22, 24, 26, 28, 30, 32, 34 or 36 months. These values may represent the lower limit of a range which is bounded at the upper end by a value selected from 8, 9, 10, 11, 12, 14, 16, 18, 20, 22, 24, 26, 28, 30, 32, 34, 36, 48 or 60 months.

Wherein the above methods are for preventing or reducing interaction between said biologically-active cargo and an adjuvant component of the aqueous environment, in some embodiments said adjuvant is aluminium hydroxide. In some embodiments, the cargo contains phosphate groups such as in phosphodiester moieties and/or has a low pI. In some embodiments, the cargo is Hib-TT or Hib-CRM197 or MenA-CRM197.

The above methods may alternatively be for preventing or reducing interaction between said biologically-active cargo and a second cargo. Such embodiments comprise, in addition to the above-recited steps (i) and (ii), a step (iii): forming a second plurality of particles comprising said second cargo within a matrix, wherein said second plurality of particles is as defined herein for the plurality of particles but with the proviso that the cargo is not the same as said biologically-active cargo, i.e. the second plurality of particles is as defined herein for the plurality of particles with the exception of the cargo, in the sense that in such embodiments comprising two pluralities (populations) of particles, the two pluralities comprise different cargoes. In some alternative embodiments, the two pluralities may comprise the same cargo in a different polymer matrix.

The plurality of particles and the (immunogenic) compositions and vaccines of the invention may be used in medicine, such as prophylactically or therapeutically. They may be administered to a subject in need thereof. Typically the subject is an animal, such as a mammal, and is preferably a human subject. In some embodiments, the subject is an infant or a child or an adolescent or an adult or an elderly adult. The subject may be a pregnant female, optionally wherein the gestational infant is the subject in need. The subject may be an immunocompromised individual. In a particular embodiment, the plurality of particles and the immunogenic compositions and vaccines are for use in immunisation, such as paediatric immunisation.

In one aspect the invention provides a plurality of particles or a composition/immunogenic composition/vaccine as disclosed herein for use in medicine, in particular in human medicine. More particularly, the invention provides a plurality of particles as defined herein, or a composition/immunogenic composition/vaccine as defined herein, for use in the treatment or prevention, in particular in a human, of (i) an infection or pathology caused directly or indirectly by a pathogen or allergen, or (ii) a pathology associated with immunologically distinct host cells, such as cancer.

The invention further provides the use of a plurality of particles as defined herein, or a composition/immunogenic composition/vaccine as defined herein, in the manufacture of a medicament for use in the treatment or prevention, in an animal, in particular in a human, of (i) an infection or pathology caused directly or indirectly by a pathogen or allergen, or (ii) a pathology associated with immunologically distinct host cells, such as cancer.

In another aspect is provided a method of treatment or prophylaxis against (i) an infection or pathology caused directly or indirectly by a pathogen or allergen, or (ii) a pathology caused by immunologically distinct host cells, such as cancer, comprising the step of administering to a subject, in particular a human, an effective amount of a plurality of particles or a composition/immunogenic composition/vaccine as disclosed herein. A method, comprising the same step of administration, is also provided for eliciting an immune response against such a pathogen or allergen or immunologically distinct host cells.

The plurality of particles or composition/immunogenic composition/vaccine of the invention may be administered in a liquid form, i.e. as a suspension containing the particles. Prior to administration, the particles may be stored in the finally-formulated, administrable liquid composition, such as the composition/immunogenic composition/vaccine of the invention in which the particles are in the aqueous environment as defined herein. However, the particles may be stored in lyophilised, such as freeze-dried, form, or powdered form, to be reconstituted into liquid form through mixing with the aqueous environment (as defined herein) extemporaneously with administration to a subject. Alternatively, the particles may be stored in liquid medium other than said aqueous environment, such that mixing with said aqueous environment, to give the composition/immunogenic composition/vaccine of the invention, takes place extemporaneously with administration. The particles or composition/immunogenic composition/vaccine of the invention may be presented in unit-dose or multi-dose sealed containers such as vials, or may be pre-filled into administration devices such as syringes.

The plurality of particles or composition/immunogenic composition/vaccine of the invention may be delivered to a subject through various routes, such as intramuscularly, intravenously, intraperitoneally, intra- or trans-dermally, subcutaneously, intrapulmonary (e.g. inhalation), trans- or sub-mucosally. Thus, in some embodiments, administration is via a parenteral route.

An appropriate effective dose of the biologically-active cargo, e.g. an antigen (and therefore, of the particles and the composition in which they are present), may readily be determined by the person skilled in the art. Such a dose can be calculated based on delivery of a specified mass of particles. In that case, a specific mass of the particle-containing composition is measured by weight or volume. Alternatively, the dose may be based on delivering a specified mass of cargo, in which case the loading of the cargo in the particles must be taken into account.

In a particular embodiment of the above plurality of particles, compositions/immunogenic compositions/vaccines; use of such; or method of treatment or of eliciting an immune response, the pathogen is selected from the list consisting of: Haemophilus influenzae type b (Hib); Neisseria meningitidis (in particular serotypes A, C, W and/or Y); Streptococcus pneumoniae; Staphylococcus aureus, Bordetella sp; and Salmonella typhi. In preferred embodiments, the pathogen is Haemophilus influenzae type b (Hib) or Neisseria meningitidis serotype A (MenA).

### Fabrication and Formulation of Particles

The particles of the present invention comprise a biologically-active cargo within a matrix. The cargo may associate with the particle matrix in various ways such that it is ‘comprised within the matrix’. For example, the cargo may be encapsulated within the matrix, or the cargo may be dispersed within or physically blended with the matrix. The combination of cargo and matrix may be described as a physical association, such as a non-covalent association.

In particular embodiments the cargo is substantially homogeneously dispersed throughout the matrix of the particle. This may be achieved by fabricating particles from a homogeneous mixture (a solution) comprising the cargo and matrix polymer(s).

Thus in one aspect is provided a method for making a plurality of drug delivery particles comprising a biologically-active cargo within a matrix, comprising the step of making a solution (i.e. homogeneous mixture) comprising said cargo and a matrix polymer, optionally wherein said polymer is as defined herein for the polymeric matrix. Also provided is a method for making a plurality of drug delivery particles comprising a biologically-active cargo within a matrix, wherein the method comprises the use of a solution comprising a polymer as defined herein for the polymeric matrix, and a cargo, wherein said cargo is optionally as defined herein.

In some embodiments of the above methods, said solution comprises said cargo at an amount not exceeding 30 wt % and a balance of PMMA-co-PMAA copolymer. For example, the solution may comprise the cargo at an amount of 0.1-5 wt %, such as 0.1, 0.2, 0.3, 0.4, 0.5, 0.6, 0.7, 0.8, 0.9, 1, 1.5, 2, 2.5 or 5 wt %. In some embodiments, the solution further comprises a plasticiser. In some embodiments the plasticiser is polyvinylpyrrolidone (PVP). In a particular embodiment the matrix polymer is PMMA-co-PMAA and the solution further comprises PVP, wherein the wt % ratio of PVP:PMMA-co-PMAA does not exceed 1:1, for example the wt % ratio of PVP:PMMA-co-PMAA is in the range 0.1-1:1, such as 0.15:1, 0.2:1, 0.25:1, 0.3:1, 0.35:1, 0.4:1, 0.45:1, 0.5:1, 0.55:1, 0.6:1, 0.65:1, 0.7:1, 0.75:1, 0.8:1, 0.85:1, 0.9:1, or 0.95:1. The PVP may, for example, have a molecular weight (Mw) of about 2.5 kDa and a polydispersity of approximately 1.9. In other embodiments of such methods, the solution comprises poly(glutamic acid)-co-poly(lysine) copolymer instead of PMMA-co-PMAA copolymer and/or glycerol instead of PVP.

The cargo may, for example, be selected from Hib-TT, Hib-CRM197 and MenA-CRM197. In particular embodiments, said solution comprises 0.2-1.2, more particularly 0.4-1, wt % Hib-TT or Hib-CRM197 or MenA-CRM197 and a balance of PVP:PMMA-co-PMAA or poly(glutamic acid)-co-poly(lysine):glycerol, optionally in 1:1 wt % ratio.

The particles of the invention may be fabricated from such solutions through known techniques for fabricating micro- or nanoparticles, for example homogenisation (emulsification), extrusion and drying such as spray-drying. In particular, the particles are formed by moulding. Hence, in some embodiments, the above methods comprise a further step of moulding said solution to form the plurality of particles. The solution may comprise a plasticiser other than PVP (optionally in addition to PVP); such plasticiser may be a porogen. In some embodiments, the methods comprise removing substantially all of said PVP or other plasticiser and/or a porogen from said particle. By ‘removing from said particle’ in this sense is meant that plasticiser and/or porogen may be substantially absent from the particles formed from said (plasticiser- and/or porogen-containing) solution as a result of the method, i.e. that the resulting particle may be substantially free of any plasticiser and/or porogen. It does not necessarily mean that the formed particles will temporarily contain such plasticiser and/or porogen (the plasticiser and/or porogen may be lost as a consequence of the particle fabrication process), though this may be the case for particles which are collected in a dry state, wherein the plasticiser and/or porogen is lost from the particle during a subsequent step of ‘transitioning’ the particles to a parenteral administration-acceptable sub-physiological pH. Inevitably, the composition of particles, after loss or removal of any plasticiser and/or porogen, will differ from the composition of the solution used in their fabrication.

In another aspect the present invention provides a method for making a plurality of drug delivery particles comprising a biologically-active cargo within a matrix, comprising the steps of:
i. at least partially deprotonating a polymer, which is insoluble in its protonated state, in an aqueous environment such that the polymer has a net negative charge and is soluble in said aqueous environment;
ii. combining said polymer with said cargo to produce a solution;
iii. forming particles by moulding said solution and removing the aqueous environment.

In the interests of clarity, it is to be noted that “aqueous environment” as used in the above paragraph is not to be confused with the use of this term elsewhere herein in relation to the compositions of the invention or the properties of the particles of the invention when present in a aqueous environment below the trigger pH.

In step (i) above, a polymer is solubilised through (at least partial) deprotonation, for example by adding the polymer to an aqueous environment of alkaline or basic pH. The dissolved polymer is then combined with the cargo to produce a solution, often referred to as a “stock solution”. The solution is then moulded as described above in connection with the PRINT™ Technology of co-applicant Liquidia Technologies and as exemplified in the Examples herein, as a result of which the aqueous environment is removed. This step (iii) of the above method causes the chains of the polymer to non-covalently associate with, or physically entangle, the cargo. In alternative embodiments, the particles may be formed not by moulding but by other known micro- or nano-particle fabrication techniques, for example spray-drying.

After formation of the particles, they may be collected in a dry state, or in an organic or aqueous liquid collection environment. Such collection environment may be acidic, for example with a pH of less than 5. In some embodiments, the above method may further comprise protonating the matrix polymer of the collected particles such that the polymer returns to an insoluble state. The particles may then be stored at a sub-physiological pH which is acceptable for parenteral delivery, for example in an aqueous environment.

In another aspect, the invention provides a plurality of drug delivery particles obtainable or obtained by the foregoing methods.

In a further aspect is provided a method for making a composition, comprising making a plurality of particles according to a method as defined herein and formulating said particles in an aqueous environment. In some embodiments, wherein the composition is an immunogenic composition, said environment comprises an antigen and/or an adjuvant. Said antigen and/or adjuvant may, for example, be as defined herein in relation to the immunogenic compositions of the invention. The invention furthermore provides, in another aspect, a method for making a composition, such as an immunogenic composition, comprising a plurality of drug delivery particles comprising a biologically-active cargo within a matrix, wherein the matrix comprises a polymer, comprising the steps of:
i. introducing a plurality of particles, made according to a method as defined herein, to an acidic aqueous environment such that the acidic environment protonates the matrix polymer making the polymer insoluble in said environment;
ii. raising the pH of the acidic aqueous environment to a sub-physiological pH which is acceptable for parenteral administration while retaining the insoluble state of the matrix polymer in said environment; and optionally
iii. formulating said particles in an aqueous environment of sub-physiological pH.

In step (i) of this method, referred to as herein as “stabilisation”, the particles are brought into contact with a “stabilising solution” at acidic pH, resulting in protonation of the matrix polymer making the polymer insoluble. For example, for particles comprising matrix polymer initially containing COOH groups predominantly in the ionised, negatively-charged (i.e. COO—) form, such protonation alters the balance in favour of the charge-neutral COOH form. The protonation of the matrix polymer causes the particles to become insoluble in the stabilising solution. The pH of the stabilising solution should be such that when bringing the particles into contact with the stabilising solution the rate of protonation exceeds the rate of dissolution. In some embodiments, the stabilising solution has a pH in the range 1-5, such as about or exactly pH 3.5 or 4.5. Such ‘bringing into contact’ may, for example, be by sprinkling dry particles into the stabilising solution while continuously stirring the latter. The duration of such contact (i.e. before step (ii) begins) may be up to 60, 50, 40, 30, 20, 10 or 5 minutes, or these values may respectively delineate the upper end of a time range which is bounded at the lower end by 1 minute.

In step (ii), known herein as “neutralisation”, the environment containing the insoluble particles is adjusted to a pH which is compatible with parenteral administration. Such pH clearly must be below the threshold pH above which the particles would release their cargo, i.e. the pH must be sub-physiological. Both ‘sub-physiological’ and ‘acceptable for parental administration’ in this sense must be with respect to the particular target administration site of/route into the particular intended subject. This adjustment in pH, for example by continuous or step-wise addition of a solution of higher pH (for example by addition of a buffer which is less acidic than the stabilising solution), must be done at an appropriate rate which maintains the insoluble state of the matrix polymer. Thus in some embodiments, step (ii) comprises increasing the pH of the aqueous environment towards a sub-physiological pH in a stepwise manner. In some embodiments, the pH of the aqueous environment is increased by 0.1-10 pH units per minute, such as 0.5, 1, 2 or 5 pH units per minute, in particular 0.5 pH units per minute. By way of clarification, this means that in every minute elapsing between the first adjustment and the attainment of the finally-adjusted sub-physiological and parenterally-acceptable pH, the increase in solution pH is in the range of 0.1-10 pH units. The combined steps of stabilisation and neutralisation are termed herein as “transition”.

In optional step (iii) the particles are formulated in an aqueous environment of sub-physiological pH, i.e. the particle-containing solution at sub-physiological pH is combined with other components to produce a composition, which composition must also be at sub-physiological pH in order that the particles retain the cargo during storage. Such “other components” present within the aqueous composition of step (iii) may include formulation excipients such as buffers, tonicity modifiers, preservatives, adjuvants, etc, as well as active ingredients such as drug compounds or antigens. In particular, the aqueous environment of step (iii) may be as defined herein for the compositions of the invention. In another aspect the invention provides a composition, such as an immunogenic composition, obtainable or obtained by the foregoing methods.

The following Examples are provided to illustrate certain particular features and/or embodiments. These Examples should not be construed to limit the invention to the particular features or embodiments described.

### EXAMPLES

### Example 1—Preparation of Particles and Particle-Containing Formulations

Particle Fabrication:

Drug delivery particles were manufactured. First, a series of stock solutions were prepared. A homogeneous aqueous solution of approximately 10 wt % PMMA-co-PMAA (Mw˜125 kDa, MMA:MAA=2:1 molar ratio, Eudragit® S100, Evonik Industries) was made by dissolving the polymer at a pH of approximately 8. A homogeneous aqueous solution of approximately 15 wt % PVP (2.5 kDa, Polysciences, Inc.) was prepared. The concentration of the Haemophilus influenzae type b polysaccharide-tetanus toxoid conjugate (“Hib-TT”) solution was 0.0946 wt % in water.

The following volumes were combined to produce the particle stock solution: 19.000 mL PMMA-co-PMAA, 12.667 mL PVP, 26.540 mL Hib-TT, and 2.111 mL WFI water. Based on solid components the particle stock solution had the following weight percent ratio: 49.67 wt % PMMA-co-PMAA, 49.67 wt % PVP, and 0.66 wt % Hib-TT. The resulting stock solution was cast at room temperature onto a 0.005 inch (0.127 mm) thick PET film using a #10 Mayer rod. To form drug delivery particles, the film was pre-laminated against a 6 μm donut-shaped PRINT® (Liquidia Technologies, Inc., Morrisville, N.C.) mould. The mould was then filled by passing through a laminator at 290° F. at 2 feet per minute. The drug delivery particles were removed from the film by mechanical scraping under dry conditions (i.e. less than 30% relative humidity) using a doctor blade.

Transition of Particles:

Under dry conditions (10-30% relative humidity), approximately 1.000 g of drug delivery particles were sprinkled onto 40 mL of rapidly stirring 0.2 M sodium succinate pH 3.5/PEG400, 50/50 by volume. The particles were stirred about 10 minutes. After 10 minutes, 4×20 mL 200 mM sodium maleate pH 6.1 aliquots were added to the suspension, with 1-2 minutes of stirring between each aliquot addition.

The suspension was divided into four polycarbonate tubes, approximately 30 mL per tube. The suspension was pelleted by spinning at 18,000×g for approximately 10 minutes at 4° C. The supernatant was removed and discarded. Each pellet was resuspended in 15 mL 200 mM sodium maleate pH 6.1 and two tubes were combined into one tube. The suspension was pelleted by spinning at 18,000×g for approximately 10 minutes at 4° C. The supernatant was removed and discarded. The pellet was resuspended in 30 mL 200 mM sodium maleate pH 6.1. The suspension was pelleted by spinning at 18,000×g for approximately 10 minutes at 4° C. The supernatant was removed and discarded. The pellet was resuspended in 30 mL 10 mM sodium maleate pH 6.1. The particle suspension was diluted by combining 15 mL of the suspension with 30 mL 10 mM sodium maleate pH 6.1.

The particle suspension was filtered through 41 μm nylon net in 10 mM sodium maleate pH 6.1. The filtrate was concentrated by spinning the suspension at 19,000×g for approximately 20 minutes at 4° C. The concentrated filtrate was pelleted by spinning at 19,000×g for approximately 20 minutes at 4° C. The supernatant was removed and discarded. The pellet was resuspended in 40 mL 10 mM sodium maleate pH 6.1 for a wash. The particles were washed two more times for a total of three washes. The particles were pelleted again and resuspended in a small volume (˜3 mL per tube) of 10 mM sodium maleate pH 6.1.

The particle content was determined gravimetrically to be 25.925 mg/mL. The total (conjugated+free) Hib content was determined by HPAEC-PAD to be 37.750 μg/mL.

Formulation of Particles:

The above particle suspension was formulated as follows.

To produce a sample to co-administer with Infanrix™ Penta (GSK Vaccines), 700-65, 8 mL of 10 mM sodium maleate/300 mM NaCl/0.02% Thimerosal pH 6.1 was dispensed into a container. 8 mL of the particle suspension described above was added. 62.297 mL of 10 mM sodium maleate/150 mM NaCl/0.01% Thimerosal, pH 6.1 was added resulting in a particle concentration of approximately 2.649 mg/mL.

To produce an Infanrix™ Penta-containing sample, 700-66, 6 mL of the particle suspension described above was pelleted. The pellet was resuspended in 1.021 mL 10 mM sodium maleate pH 6.1 and 2.669 mL 210 mM sodium maleate/0.22% Thimerosal pH 6.1. 5 mL of the resuspended particles were removed and 50 mL Infanrix™ Penta was added to the 5 mL.

To produce a second Infanrix™ Penta-containing sample, [700-83-02], 5.0 mL of the particle suspension described above was pelleted. The pellet was resuspended in 0.787 mL 10 mM sodium maleate pH 6.1 and 2.224 mL 210 mM sodium maleate/0.22% Thimerosal pH 6.1. 3 mL of the resuspended particles were removed and 30 mL Infanrix™ Penta was added to the 3 mL.

The formulations were aliquoted into vials and stored at 4° C. until use. As used in these Examples, the liquid media in which the particles of the respective samples were resuspended shall be referred to as “storage buffer”.

### Example 2—Stability of Example 1 Formulated Particles

HPAEC-PAD:

HPAEC-PAD quantification was conducted on samples containing drug delivery particles formulated in storage buffer as per Example 1. Samples were analyzed for total oligo/polysaccharide, which encompassed both conjugated (Hib-TT or Hib-CRM) and unconjugated (‘free’) oligo/polysaccharide (‘Hib’). Some samples were also analyzed for free Hib.

In the analysis of particle-containing samples, particularly those not containing aluminium adjuvant, both the drug delivery particles and the storage buffer were analyzed for total Hib (conjugated+free). In these cases, the drug delivery particles were recovered from the storage buffer using centrifugation. The storage buffer (supernatant) was removed and retained for analysis. The drug delivery particles (pellet) were re-suspended in the same volume of storage buffer that was recovered in the supernatant. The re-suspended drug delivery particles were then triggered to ‘release’ cargo by the addition of base to produce a triggered drug particle suspension. Prior to analysis, to the naked eye, the sample was clear. These samples were analyzed for both total and free Hib for both the pellet and the supernatant.

For example, 500 μL of a sample having a particle concentration of approximately 2 mg/mL was centrifuged to separate the drug delivery particles from the storage buffer. The supernatant was measured, removed, and retained for analysis. A volume of 10 mM sodium maleate pH 6.1 equal to the volume of supernatant removed was added to the pellet to re-suspend the drug delivery particles. To trigger/dissolve the drug delivery particles, 4-7 μL 0.5 N sodium hydroxide was added to the re-suspended particles to raise the pH to approximately 7.0-7.5, targeting approximately 7.2. The volume of base added was adjusted as needed to maintain the ratio of base to particles as provided in this Example. For some samples, particularly those containing aluminium adjuvant, total Hib was determined. In these cases, the drug delivery particles were not recovered from the storage buffer using centrifugation. The sample was triggered by the addition of base to produce a triggered drug particle suspension. Prior to analysis, to the naked eye, the sample was clear. These samples were analyzed for total Hib. This measure of total Hib reflected the total Hib contained in the sample (both drug delivery particles and storage buffer).

For example, 500 μL of a sample having a particle concentration of approximately 2 mg/mL was triggered by the addition of base. To the sample, 4-7 μL 0.5 N sodium hydroxide was added to raise the pH of the sample to approximately 7.0-7.5, targeting approximately 7.2. The volume of base added was adjusted as needed to maintain the ratio of base to particles as provided in this example.

Standard/Control Preparation:

Standards: Hib standards were prepared in the concentration range of interest. For example, standards at 0.625 μg/mL, 1.25 μg/mL, 2.50 μg/mL, 5.00 μg/mL, 10.0 μg/mL, 15.0 μg/mL, 20.0 μg/mL, and 25.0 μg/mL were prepared by diluting a known concentration of Hib using 10 mM sodium maleate pH 6.1.

Control: Optionally, control samples can be included in the analysis. For example, HIBERIX™ [Haemophilus b Conjugate Vaccine (Tetanus Toxoid Conjugate)] having a known concentration may be included as a control sample. If using HIBERIX™, a first control sample can be produced by reconstituting the lyophilized vaccine using 0.9% NaCl as described in the Prescribing Information. To produce a second control sample, a portion of the reconstituted vaccine is then diluted ten-fold using additional 0.9% NaCl. Both control samples may be analyzed.

Analysis of Free Polysaccharide:

To analyze free oligo/polysaccharide, deoxycholate was used to precipitate and remove the conjugated polysaccharide from the sample of interest.

A 1% (m/v) solution of deoxycholate (DOC) in deionised water was prepared in advance and stored at −20° C. in aliquots for no more than six months. Approximately 1 g of deoxycholate was added to 90 mL deionised water with agitation. After the deoxycholate was completely dissolved, the pH of the solution was slowly increased to 6.8 by dropwise addition of 1M sodium hydroxide. Precipitate formed upon addition of each drop of base was allowed to dissolve prior to addition of another drop of base. After the pH was adjusted, deionised water was added to bring the final volume to 100 mL. To prepare a sample for analysis, 100 μL of supernatant or triggered drug particle suspension (from the pellet that was resuspended) was placed into a 1.5 mL LoBind Microcentrifuge Tube (Eppendorf) and 100 μL of the 1% DOC solution was added and the tube was vortexed to ensure uniform mixing. The sample was cooled on ice for approximately 30 minutes. After 30 minutes, 10 μL 1N HCl was added and the sample was vortexed. Conjugated oligo/polysaccharide, Hib-TT or Hib-CRM, bound to the DOC creating a precipitate. The precipitate was removed using centrifugation at 19,500×g for 15 minutes at approximately 4° C. and room temperature. The pellet was discarded and the supernatant was retained for analysis of free Hib.

Sample Preparation: Hydrolysis to Ribitol Ribose 5-Phosphate:

Samples were hydrolyzed and produced ribitol ribose 5-phosphate as the analyte of interest.

Standards/Control: To 100 μL of standard or control, 150 μL 1N sodium hydroxide was added. The sample was vortexed to mix the base into the sample uniformly. The sample hydrolyzed at room temperature for approximately 12 hours.

Samples of Interest: To 100 μL of sample (total sample oligo/polysaccharide, total pellet polysaccharide, total supernatant polysaccharide, free pellet polysaccharide, or free supernatant polysaccharide), 150 μL 1N sodium hydroxide was added. The sample was vortexed to mix the base into the sample uniformly. The sample hydrolyzed at room temperature for approximately 12 hours.

Instrument Parameters

| Parameter | | |
|---|---|---|
| Mobile Phase | 100 mM sodium acetate/ | |
| | 30 mM sodium hydroxide | |
| Column System | Dionex AminoTrap | |
| | Trap Column, 4 × 50 mm | |
| | Dionex CarboPac PA10 | |
| | Guard Column, 4 × 50 mm | |
| | Dionex CarboPac PA10 | |
| | Analytical Column, 4 × 250 mm | |
| Column | 35° C. | |
| Temperature | | |
| Flow Rate | 1.00 | mL/min, isocratic |
| Injection volume | 40 | μL |
| Auto Sampler | 4° C. | |
| Temperature | | |
| Run Time | 35 minutes | Pellet or |
| | | supernatant samples |
| | 60 minutes | Total system samples |
| | 80 minutes, | Hiberix Controls |
| | followed | |
| | by 60 minute | |
| | buffer blank | |
| | injection | |
| PAD Range Setting | 100 | nC |

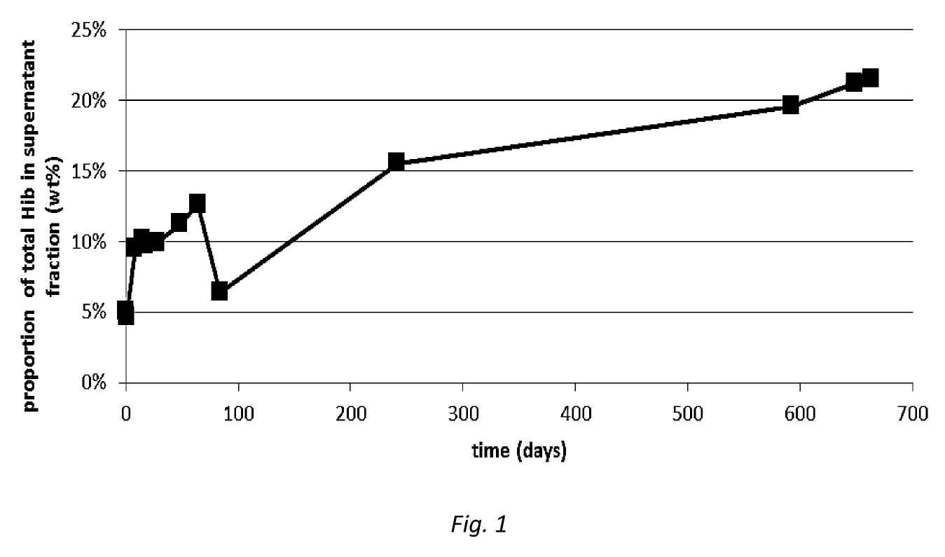

Sample concentrations were calculated by comparing the peak area of ribitol ribose 5-phosphate in the sample of interest to a least squares linear regression fit of the standards. Concentration was reported as μg/mL. Vials containing sample 700-65, were stored at 4° C. and the distribution (i.e. particle-associated, or not) of Hib-TT conjugate and unconjugated (‘free’) Hib was evaluated over time (T=0, 1, 8, 15, 18, 28, 49, 64, 84, 242, 593, 649, and 663 days) using HPAEC-PAD. Table 1 shows the proportion (in wt %) of total (i.e. conjugated+free) Hib, as measured at each timepoint, which is found in the pellet fraction (i.e. particle-associated) and in the supernatant fraction, i.e. not particle-associated. FIG. 1 shows the portion of total (conjugated+free) Hib found in the supernatant fraction, i.e. not particle-associated.

TABLE 1

| | Pellet- | Supernatant- |
|---|---|---|
| | total Hib | total Hib |
| | (Hib-TT + | (Hib-TT + |
| Time | free Hib) | free Hib) |
| Days | wt % | wt % |
| 0 | 94.94% | 5.06% |
| 1 | 95.34% | 4.66% |
| 8 | 90.46% | 9.54% |
| 15 | 89.79% | 10.21% |
| 18 | 90.29% | 9.71% |
| 28 | 90.02% | 9.98% |
| 49 | 88.72% | 11.28% |
| 64 | 87.35% | 12.65% |
| 84 | 93.56% | 6.44% |
| 242 | 84.43% | 15.57% |
| 593 | 80.39% | 19.61% |
| 649 | 78.75% | 21.25% |
| 663 | 78.46% | 21.54% |

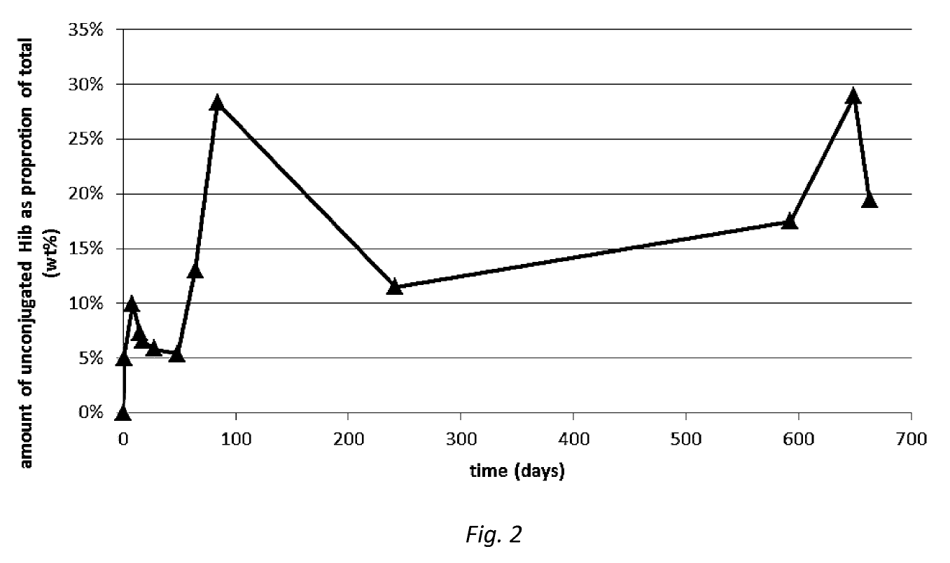

Table 2 and FIG. 2 show the proportion of free (unconjugated) Hib (in wt %) as a percentage of total (i.e. conjugated+free) Hib contained in the sample (collectively in pellet and supernatant), as measured at each timepoint. At each timepoint, both total (conjugated and unconjugated) Hib and free Hib (unconjugated) were determined for the pellet (particle) and supernatant.

TABLE 2

| | Free Hib |
|---|---|
| | (proportion |
| Time | of total Hib) |
| Days | wt % |
| 0 | 0.00% |
| 1 | 5.02% |
| 8 | 9.94% |
| 15 | 7.24% |
| 18 | 6.54% |
| 28 | 5.81% |
| 49 | 5.42% |
| 64 | 13.09% |
| 84 | 28.34% |
| 242 | 11.51% |
| 593 | 17.51% |
| 649 | 28.92% |
| 663 | 19.36% |

pH:

The pH of samples 700-65 and 700-66 were also monitored over time. A 200 to 300 μL aliquot of test sample was dispensed into a 1.5 mL tube and pH was determined using a pH meter (Hach Corporation, Model IQ 150) with an ISFET probe (Hach Corporation, pH 17.SS). Prior to determining the pH, the meter was calibrated using pH 4.01 (Orion 910104) and pH 7.00 (Orion 910107) standards. Table 3 shows the pH values collected at several time intervals (NA=sample not analysed).

TABLE 3

| Time | 700-65 | 700-66 |
|---|---|---|
| Days | pH | pH |
| 0 | 6.01 | NA |
| 1 | 5.99 | NA |
| 8 | 6.01 | NA |
| 14 | 5.96 | 5.97 |
| 28 | 5.93 | NA |
| 49 | 5.95 | NA |
| 64 | 6.01 | NA |
| 84 | 5.97 | NA |
| 648 | 5.96 | 5.99 |

### Example 3—Preparation of Further Particles and Particle-Containing Formulations

Particle Fabrication:

Drug delivery particles were manufactured as for Example 1, except that the concentration of the Hib-TT stock solution was 0.0957 wt % in water.

Transition of Particles:

Under dry conditions (20-30% relative humidity), approximately 800 mg of drug delivery particles were sprinkled onto 40 mL of rapidly stirring 0.1 M sodium succinate pH 4.5 buffer/PEG400, 50/50 by volume. The particles were stirred about 5 to 10 minutes. After 5-10 minutes, 4×20 mL 0.2 M sodium maleate pH 6.1 aliquots were added to the suspension, with 1-2 minutes of stirring between each aliquot addition.

The transition was continued as for Example 1, with minor modifications: after dividing the suspension into four polycarbonate tubes it was pelleted by spinning at 18,000×g for at least 15 minutes at 4° C., and; the subsequent centrifugation was performed at 18,000×g for 5 minutes.

The particle content was determined gravimetrically to be 14.12 mg/mL. The total Hib (conjugated+free) content was determined to be 39.595 μg/mL using HPAEC-PAD.

Formulation of Particles:

The above particle suspension was formulated as follows.

Sample 841-57-1 was produced for co-administration with Infanrix™ Penta. To produce sample 841-57-1, 7 mL volume of the particle suspension described above was diluted using 7 mL 10 mM sodium maleate/300 mM sodium chloride/0.01% thimerosal, pH 6.1 and 25.136 mL 10 mM sodium maleate pH 6.1 resulting in a particle concentration of 2.526 mg/mL.

Sample 841-57-2 was produced. To produce sample 841-57-2 11 mL of the particle suspension described above was pelleted. The pellet was resuspended in 0.878 mL 10 mM sodium maleate pH 6.1 and 2.795 mL 210 mM sodium maleate/0.21% thimerosal pH 6.1. 5 mL of the resuspended particles were removed and 50 mL Infanrix™ Penta was added to the 5 mL.

The particle-containing samples were aliquoted into vials for various in vivo studies and stability studies. Sample 841-57-2S was produced by aliquoting sample 841-57-2 into syringes for a stability study. The formulations were stored at 4° C. until use.

### Example 4—Stability of Example 3 Formulated Particles

HPAEC-PAD:

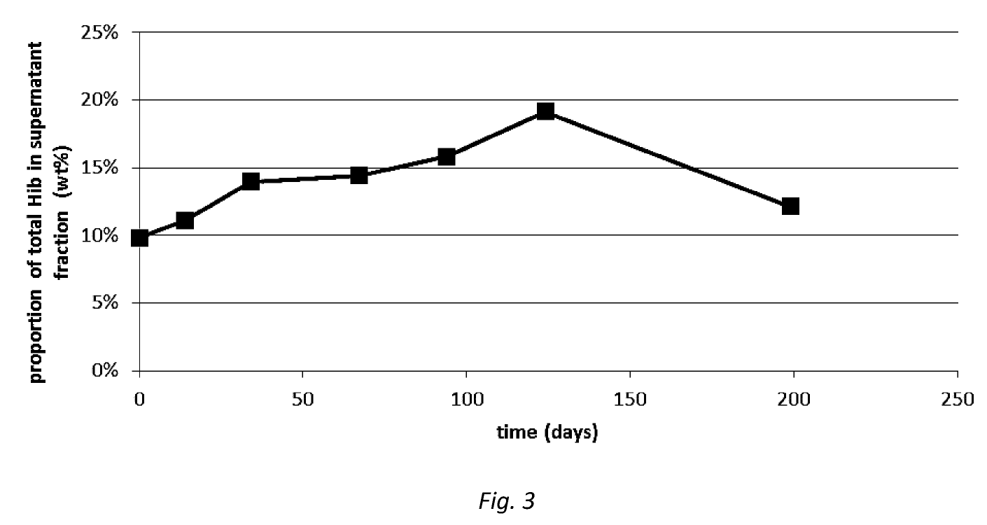

Vials containing the Example 3 samples were stored at 4° C. and the distribution (i.e. particle-associated, or not) of Hib-TT conjugate and unconjugated (‘free’) Hib was evaluated over time (T=0, 14, 34, 67, 94, 124 and 199 days) using HPAEC-PAD, as described for Example 2. For sample 841-57-1 (Hib-TT-containing particles stored in buffer at pH 6.1) Table 4 shows the proportion (in wt %) of total (i.e. conjugated+free) Hib, as measured at each timepoint, which is found in the pellet fraction (i.e. particle-associated) and in the supernatant fraction, i.e. not particle-associated. FIG. 3 shows the portion of total (conjugated+free) Hib found in the supernatant fraction, i.e. not particle associated.

TABLE 4

| | Pellet- | Supernatant- |
|---|---|---|
| | total Hib | total Hib |
| | (Hib-TT + | (Hib-TT + |
| Time | free Hib) | free Hib) |
| Days | wt % | wt % |
| 0 | 90.18% | 9.82% |
| 14 | 88.87% | 11.13% |
| 34 | 86.04% | 13.96% |
| 67 | 85.58% | 14.42% |
| 94 | 84.16% | 15.84% |
| 124 | 80.84% | 19.16% |
| 199 | 87.84% | 12.16% |

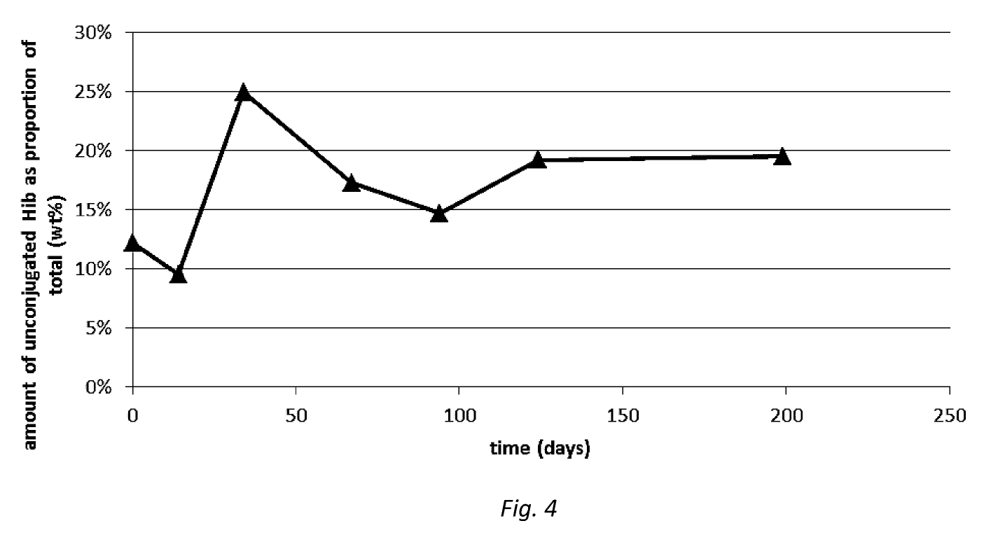

Table 5 and FIG. 4 show the proportion of free (unconjugated) Hib (in wt %) as a percentage of total (i.e. conjugated+free) Hib as contained in the sample (collectively in pellet and supernatant), as measured at each timepoint. At each timepoint, both total (conjugated and unconjugated) Hib and free Hib (unconjugated) were determined for the pellet (particles) and supernatant.

TABLE 5

| | Free Hib |
|---|---|
| | (proportion |
| Time | of total Hib) |
| Days | wt % |
| 0 | 12.22% |
| 14 | 9.55% |
| 34 | 24.98% |
| 67 | 17.29% |
| 94 | 14.74% |
| 124 | 19.25% |
| 199 | 19.57% |

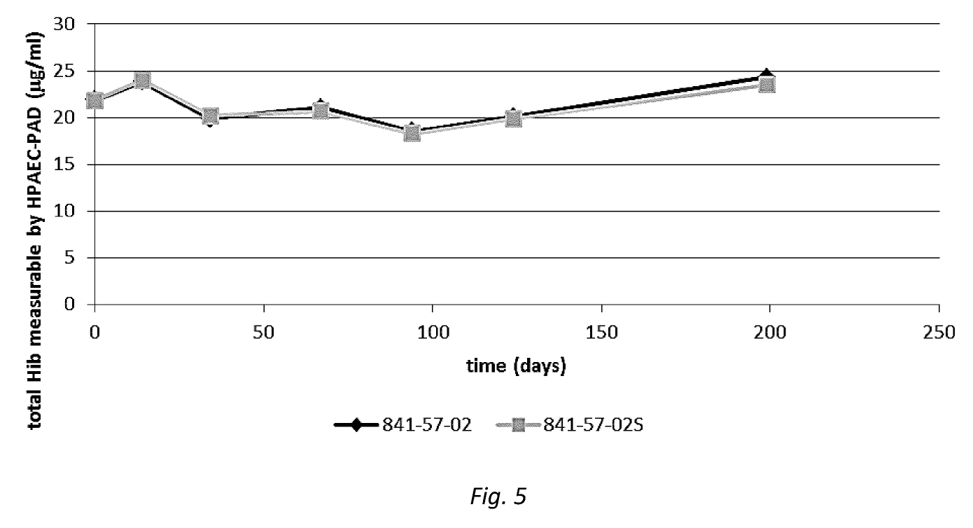

For samples 841-57-2 and 841-57-2S (Hib-TT-containing particles stored in Infanrix™ Penta, the latter in a syringe), and 841-57-1 discussed above, Table 6 and FIG. 5 show the concentration (μg/mL) of total (i.e. conjugated+free) Hib measured using HPAEC-PAD at the same timepoints. The reported concentration reflects the total Hib in the sample (particles as well as aqueous environment).

TABLE 6

| | 841-57-2 | 841-57-2S | 841-57-1 |
|---|---|---|---|
| | Total Hib | Total Hib | Total Hib |
| | (Hib-TT + | (Hib-TT + | (Hib-TT + |
| Time | free Hib) | free Hib) | free Hib) |
| Days | μg/ml | μg/ml | μg/ml |
| 0 | 21.90 | 21.86 | 22.07 |
| 14 | 23.84 | 24.00 | 23.92 |
| 34 | 19.87 | 20.18 | 20.50 |
| 67 | 21.09 | 20.70 | 20.53 |
| 94 | 18.51 | 18.33 | 20.01 |
| 124 | 20.13 | 19.91 | 22.60 |
| 199 | 24.34 | 23.52 | 22.51 |

### Example 5—Particle Responsiveness to pH Trigger

Cargo Release Around Threshold pH

Particle Fabrication:

Drug delivery particles were manufactured. First, a series of stock solutions were prepared. A homogeneous aqueous solution of 10 wt % PMMA-co-PMAA (Mw˜125 kDa, MMA:MAA=2:1 molar ratio, Eudragit® S100, Evonik Industries) was made by dissolving the polymer at a pH of approximately 8.0. A homogeneous aqueous solution of 15 wt % PVP (2.5 kDa, Polysciences, Inc.) was prepared. The concentration of the Hib-TT solution was 0.0902 wt % in water.

The following volumes were combined to produce the particle stock solution: 17.325 mL PMMA-co-PMAA, 11.550 mL PVP, 25.575 mL Hib-TT, and 0.550 mL WFI water. Based on solid components, the particle stock solution had the following weight percent ratio: 49.67 wt % PMMA-co-PMAA, 49.67 wt % PVP, and 0.66 wt % Hib-TT. The resulting stock solution was cast at room temperature onto a 0.004 inch (0.102 mm) thick PET film using a #10 Mayer rod. To form drug delivery particles, the film was laminated against a 6 μm donut-shaped PRINT® (Liquidia Technologies, Inc., Morrisville, N.C.) mould. The film/mould was passed through a laminator at 290° F. at 2 ft/min. The laminate was cooled slowly in the air. Particles, 855-51, were collected under dry conditions (20% relative humidity) using a doctor blade. Drug delivery particles 855-51 were transitioned into various vehicles to produce a series of formulations using the following procedures.

Transition of Particles:

Sample 855-61-1 was produced. Approximately 0.95 g of 855-51 was added to a tube containing 40 mL of stirring 0.2 M sodium succinate pH 3.5/PEG400 50/50 by volume. After stirring for approximately 10 minutes, 4×20 mL aliquots of 200 mM sodium maleate pH 6.1 were added with approximately one minute of stirring between additions. The suspension was divided into 4 tubes (˜30 mL per tube) and the particles were pelleted by spinning at 18,000×g at 4° C. for approximately 20 minutes. Supernatant was discarded and the particles were resuspended using 15 mL 200 mM sodium maleate pH 6.1 per tube. Four tubes were combined into two tubes and the particles were pelleted. The supernatant was removed and discarded. Each pellet was resuspended in 30 mL 200 mM sodium maleate pH 6.1. The suspension was pelleted by spinning at 19,000×g at 4° C. for approximately 10 minutes. The supernatant was removed and discarded. Each pellet was resuspended in 15 mL 10 mM sodium maleate pH 6.1 and the suspension was diluted threefold with the addition of 30 mL 10 mM sodium maleate pH 6.1 per tube.

The particle suspension was filtered through 41 μm nylon net in 10 mM sodium maleate pH 6.1. The filtrate was concentrated by spinning the suspension at 19,000×g at 4° C. for approximately 15 minutes. The concentrated filtrate was pelleted by spinning at 20,000×g at 4° C. for approximately 20 minutes. The supernatant was removed and discarded. The pellet was resuspended in 40 mL 10 mM sodium maleate pH 6.1 for a wash. The particles were washed three more times for a total of four washes. The particles were pelleted again and resuspended in a small volume (˜3 mL per tube) of 10 mM sodium maleate pH 6.1. The suspension was analyzed for particle concentration and polysaccharide content via HPAEC-PAD. The particle concentration of 855-61-1 was 28.26 mg/mL, and it contained 16.802 μg/mL total (conjugated+free) Hib.

Incubation at Varying pH:

An aliquot of sample 855-61-1, particle concentration of 28.26 mg/mL in 10 mM sodium maleate pH 6.1, was diluted to approximately 2 mg/mL using 10 mM sodium maleate pH 6.1 to produce 855-118-A.

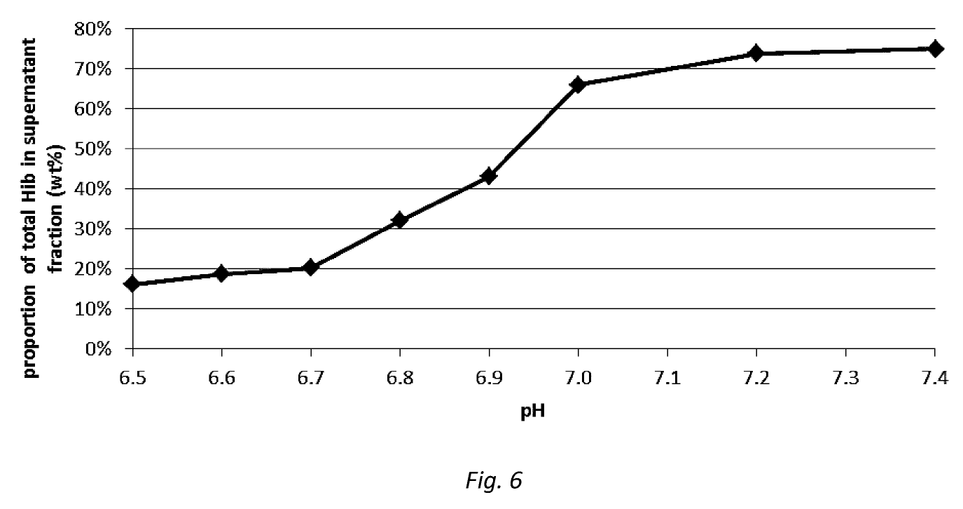

Fourteen aliquots of 0.5 mL each were dispensed into 1.5 mL tubes which were then spun at 17,000×g for approximately 20 minutes at 4° C. to pellet the drug delivery particles. The supernatant was removed and discarded. Pellets were resuspended in a series of buffers with increasing pH. Buffers were produced using 1×PBS at the following pH values: 6.5, 6.6, 6.7, 6.8, 6.9, 7.0, 7.2, and 7.4. The particles were maintained in the respective buffers at ambient temperature without agitation for approximately four hours. After four hours, the particle samples were pelleted at 17,000×g for approximately 20 minutes at 4° C. The supernatant was collected for analysis and the pellet was resuspended in 1×PBS pH 7.4, giving a clear suspension. Samples (supernatant and pellet) were analyzed for total Hib (conjugated+free) via HPAEC-PAD. Table 7 and FIG. 6 present the results, showing a marked increase in cargo release (total Hib in supernatant) as pH increases from 6.5 to 7.4.

TABLE 7

| | 855-118-A |
|---|---|
| | Supernatant-total Hib |
| | (Hib-TT + free Hib) |
| pH | wt % |
| 6.5 | 16.26% |
| 6.6 | 18.83% |
| 6.7 | 20.32% |
| 6.8 | 32.16% |
| 6.9 | 43.19% |
| 7.0 | 66.14% |
| 7.2 | 73.88% |
| 7.4 | 75.09% |

pH-Triggered Cargo Release at Varying Particle Concentration

Polymer Synthesis

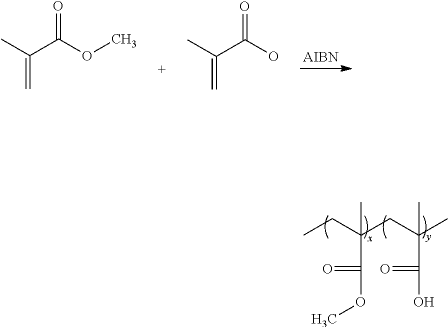

A series of PMMA-co-PMAA polymers were synthesized according to the above reaction scheme. A mole ratio target of 2:1 (MMA:MAA) was maintained. Table 8 details the raw materials used.

TABLE 8
Raw Materials

| Material | Abbreviation | Function |
|---|---|---|
| Methyl methacrylate | MMA | Reactive Monomer |
| Methyl acrylic acid | MAA | Reactive Monomer |
| 1-dodecanethiol | DDT | Chain transfer agent |
| Azobisisobutyronitrile | AIBN | Initiator (free radical) |
| Toluene (ACS Certified) | C7H8 | Reaction Medium |

Synthesis Method:

Inhibitors were removed from reactive monomer methyl methacrylate (Aldrich M55909) by passing the monomer through a column packed with inhibitor removal beads (Aldrich 306312). The procedure was repeated with methyl acrylic acid (Aldrich 155721) using a column with fresh beads. Monomers with the inhibitor removed were stored in the dark prior to use.

The volumes of the various reagents and solvents were calculated and are listed in Table 9. Materials were added to a 25 or 50 mL round bottom flask. After the reagents were added, the flask was sealed and sparged a second time with dry nitrogen for at least five minutes. A reflux condenser was added and the system was flushed with nitrogen. The flask was heated to 80° C. The reaction proceeded overnight at 80° C. with nitrogen under reflux conditions with stirring.

The following morning, the reaction was removed from heat and allowed to cool. The gelatinous product was dissolved in 15 mL ACS grade THF. The polymer was precipitated by dripping into 300 mL ice cold stirring ethyl ether (anhydrous, ACS grade). The precipitated polymer was recovered using centrifugation (3,000 g×3 minutes). The precipitate was transferred to a 50 mL Erlenmeyer flask and dissolved in 20 mL ACS grade THF. The polymer was precipitated a second time in stirring ice cold ethyl ether (anhydrous, ACS grade). The precipitated polymer was recovered using centrifugation (3,000×3 minutes) and washed with room temperature 4×50 mL ethyl ether (anhydrous, ACS grade). The polymer pellet was then transferred to a watch glass and allowed to dry under vacuum for twenty minutes.

After vacuum drying, the polymer was manually ground to a fine powder using a mortar and pestle. The polymer was transferred to a pre-weighed vial and placed into a vacuum oven at 60° C. to dry overnight. The following morning the oven was allowed to cool to room temperature while maintaining a vacuum. The resulting polymer was weighed to determine yield, assayed by GPC to determine molecular weight, and analyzed by NMR to determine MMA content.

TABLE 9
Raw Material Volumes/Masses

| Lot | MMA, | MAA, | DDT, | AIBN, | Toluene, |
|---|---|---|---|---|---|
| Number | mL | mL | uL | mg | mL |
| 816-22 | 1.068 | 0.430 | 0.0 | 4.7 | 4.3 |
| 816-33 | 1.068 | 0.430 | 6.6 | 4.7 | 4.3 |
| 831-30 | 1.160 | 0.357 | 19.8 | 4.7 | 4.3 |
| 852-2-2 | 1.068 | 0.430 | 19.8 | 4.7 | 4.3 |
| 852-4-3 | 1.068 | 0.430 | 19.8 | 4.7 | 4.3 |
| 852-4-4 | 1.068 | 0.430 | 19.8 | 4.7 | 4.3 |
| 852-9-6 | 1.068 | 0.430 | 19.8 | 4.7 | 4.3 |
| 852-13-7 | 1.068 | 0.430 | 19.8 | 4.7 | 4.3 |
| 852-13-8 | 1.068 | 0.430 | 19.8 | 4.7 | 4.3 |

Polymer Analysis: GPC and NMR:

The resulting polymers were analyzed for molecular weight using GPC and PMMA content using NMR. For the NMR analysis, percent PMMA was determined by comparing the integration of the proton signal arising from the methyl group on methacrylate to the proton signal from the acid proton on methacrylic acid. Table 10 summarizes the results of the analysis. The overall yield is also included in Table 10. Use of a chain transfer agent reduced the molecular weight of the resulting polymer. Increasing the amount of the chain transfer agent further reduced the molecular weight of the resulting polymer.

TABLE 10
Molecular Weight and PMMA by NMR Results

| | Molecular | Molecular | Molecular | | | |
|---|---|---|---|---|---|---|
| Lot | Weight | Weight | Weight | | PMMA | Yield, |
| Number | (Mₙ, Da) | (M_{W}, Da) | (M_{P}, Da) | PDI | % | mg |
| 816-22 | 63,383 | 124,235 | 103,014 | 1.96 | 62 | 977 |
| 816-33 | 39,688 | 76,765 | 73,441 | 1.93 | 61 | 537 |
| 831-30 | 33,832 | 53,038 | 52,855 | 1.56 | 65 | 857 |
| 852-2-2 | 48,122 | 61,410 | 52,410 | 1.28 | 61 | 886 |
| 852-4-3 | 51,474 | 70,280 | 63,254 | 1.37 | 62 | 934 |
| 852-4-4 | 34,643 | 46,314 | 43,457 | 1.34 | 61 | 940 |
| 852-9-6 | 35,433 | 45,829 | 50,547 | 1.29 | 61 | 919 |
| 852-13-7 | 46,436 | 66,326 | 72,142 | 1.43 | 61 | 955 |
| 852-13-8 | 39,533 | 53,666 | 54,603 | 1.36 | 59 | 882 |

Particle Fabrication:

Drug delivery particles were manufactured. First, a series of stock solutions were prepared. A homogeneous aqueous solution of 6 wt % PMMA-co-PMAA (Lots 852-2-2, 852-4-3, 852-4-4, 852-9-6, 852-13-7, and 852-13-8) was made by dissolving the polymer at a pH of approximately 8-12. A homogeneous aqueous solution of 15 wt % PVP (2.5 kDa, Polysciences, Inc.) was prepared. The concentration of the Hib-TT solution was 0.0902 wt % in water.

The following volumes were combined to produce the particle stock solution: 52.560 mL PMMA-co-PMAA, 21.024 mL PVP, 46.200 Hib-TT, and 0.216 mL WFI water. Based on solid components, the particle stock solution had the following weight percent ratio: 49.67 wt % PMMA-co-PMAA, 49.67 wt % PVP, and 0.66 wt % Hib-TT. The resulting stock solution was cast at room temperature onto a 0.005 inch (0.127 mm) thick raw PET film using a #12 Mayer rod. To form drug delivery particles, the film was laminated against a 6 μm donut-shaped PRINT® (Liquidia Technologies, Inc., Morrisville, N.C.) mould. The film/mould was passed through a laminator at 290° F. and a line speed of 2 ft/min. The laminate was cooled slowly in the air. Particles were collected under dry conditions (5-15% relative humidity) using a doctor blade. The lot number of the particles was 855-12.

Transition of Particles:

The harvested particles were divided into four samples. Under dry conditions (10-20% relative humidity), approximately 900 mg of drug delivery particles were sprinkled onto 40 mL of rapidly stirring stabilizing solution (0.2 M sodium succinate pH 3.5 buffer/PEG400, 50/50 by volume). The particles were stirred about 10 minutes. After 10 minutes of stirring the sample was moved to a sterile hood and 8×10 mL 400 mM sodium maleate pH 6.1 aliquots were added to the suspension, with 1 minute of stirring between each aliquot addition. The suspension was divided into four polycarbonate tubes, approximately 30 mL per tube. The suspension was pelleted by spinning at 18,000×g for approximately 25 minutes at 4° C. The supernatant was removed and discarded. Each pellet was resuspended in 15 mL 400 mM sodium maleate pH 6.1 and four tubes were combined into two tubes. The suspension was pelleted by spinning at 18,000×g for approximately 25 minutes at 4° C. Supernatant was removed and discarded. Each pellet was resuspended in 30 mL 200 mM sodium maleate pH 6.1. The suspension was pelleted by spinning at 19,000×g for approximately 10 minutes at 4° C. The supernatant was removed and discarded. Each pellet was resuspended in 15 mL 10 mM sodium maleate pH 6.1 and diluted with an additional 30 mL 10 mM sodium maleate pH 6.1. The particle suspension was filtered through 41 μm nylon net in 10 mM sodium maleate pH 6.1. The nylon net was rinsed with additional 10 mM sodium maleate pH 6.1 buffer. The filtrate was concentrated by dividing the material into two tubes and spinning the suspension at 19,000×g for approximately 10 minutes at 4° C. The concentrated filtrate was pelleted by spinning at 19,000×g for approximately 10 minutes at 4° C. The supernatant was removed and discarded. Each pellet was resuspended in 40 mL 10 mM sodium maleate pH 6.1 for a wash. The particles were washed two more times for a total of three washes. Particles were resuspended in approximately 3 mL 10 mM sodium maleate pH 6.1 per tube. The particle concentration of the suspension was determined to be 12.5 mg/mL. An aliquot of the suspension was tested for total Hib concentration using HPAEC-PAD. The result was 17.52 μg/mL. The particle concentration of the suspension was 12.50 mg/mL. The lot number for the particle suspension was 855-14. The sample was stored at 4° C. until further use.

Incubation at Varying pH and Particle Concentration:

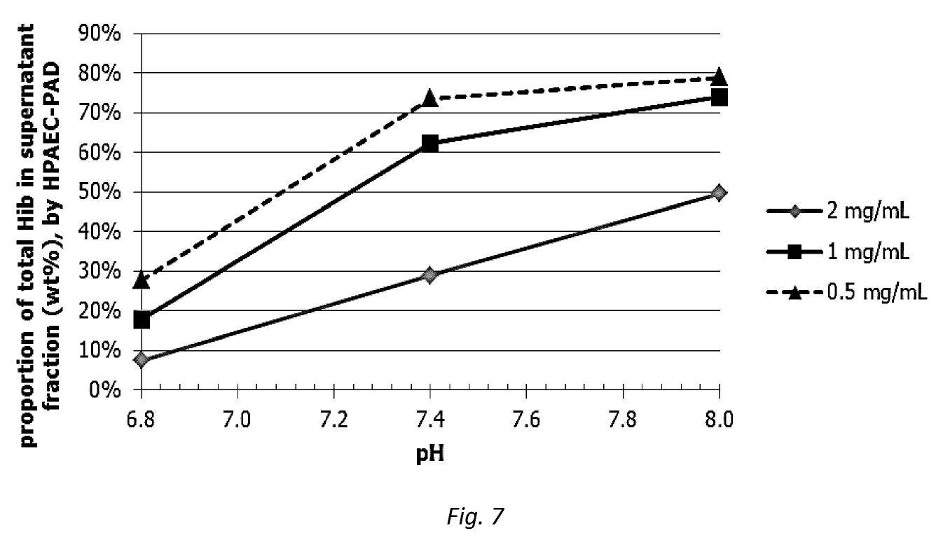

Particle formulation 855-14, with a particle concentration of 12.5 mg/mL in 10 mM sodium maleate pH 6.1, was dispensed into vials. For a sample with a particle concentration of 2 mg/mL, 80 μL was dispensed into the vial; for 1 mg/mL, 40 μL was dispensed; and for 0.5 mg/mL, 20 μL was dispensed. The dispensed aliquot was pelleted at 17,000×g for approximately 20 minutes at 4° C. to reconstitute the drug delivery particles at each particle concentration of interest. 1×PBS at pH 6.8 and pH 8.0 was prepared. 1×PBS was also used as is with pH 7.4. The 2 mg/mL pelleted samples were resuspended in pH 6.8, 7.4, and 8.0 1×PBS. The process was repeated for the 1 mg/mL and 0.5 mg/mL samples as well. After incubation for four hours without agitation at ambient conditions, the samples were pelleted at 17,000×g for approximately 20 minutes at 4° C. The supernatant was collected for analysis and the pellet was resuspended in 1×PBS pH 7.4. Samples (supernatant and pellet) were analyzed for total Hib via HPAEC-PAD. Table 11 and FIG. 7 present the results obtained for the samples at the various particle concentrations and pH values.

TABLE 11

| | Particle concentration | | |
|---|---|---|---|
| | 2 mg/mL | 1 mg/mL | 0.5 mg/mL |
| pH | HPAEC-PAD (wt %) | | |
| 6.8 | 7.53% | 17.81% | 27.78% |
| 7.4 | 28.98% | 62.34% | 73.68% |
| 8.0 | 49.60% | 74.03% | 78.95% |
| 8.0 | 67.73% | 83.87% | 79.78% |

The samples demonstrated both a particle concentration- and a pH-dependent total Hib release; for a given particle concentration, higher pH levels led to a higher percentage of Hib in the supernatant. For a given pH value, a lower particle concentration lead to a higher percentage of Hib in the supernatant.

Particle Dissolution at Threshold pH by Optical Microscopy:

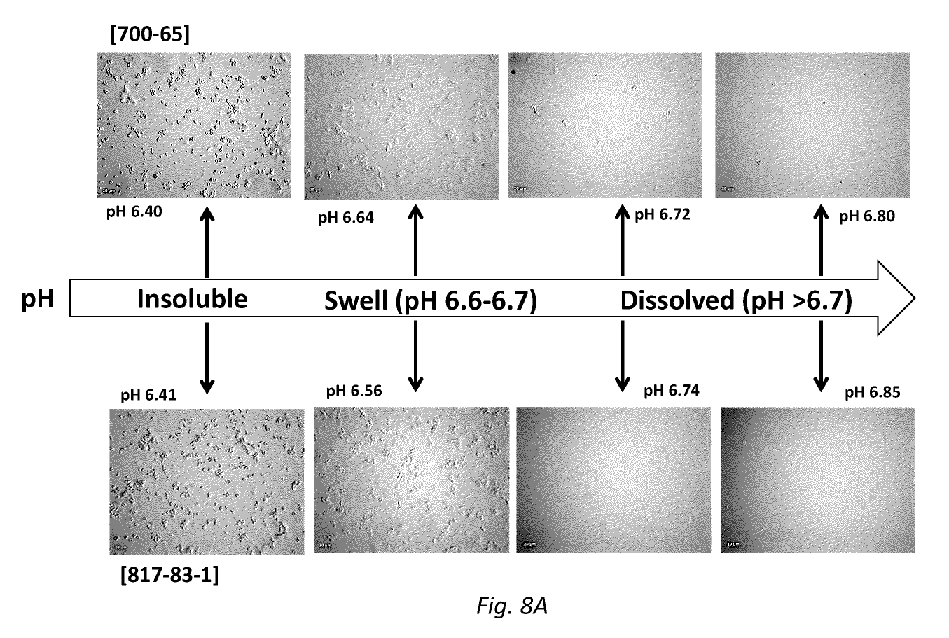

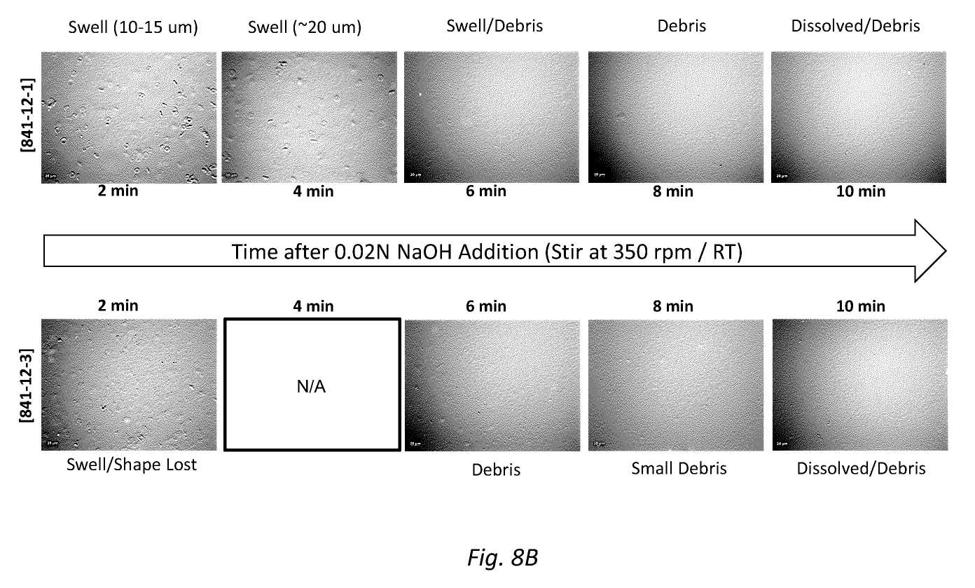

After approximately one year of storage at 4° C., 700 μL of sample 700-65 was dispensed into a 1 dram vial. 0.01 N NaOH was added incrementally using a 250 μL Hamilton syringe fitted with a 27 gauge needle. The pH was monitored using a pH meter. A small aliquot was removed periodically and was analyzed using an optical microscope. Table 12 details the data collected in the study. FIG. 8A depicts the optical images.

TABLE 12

| 0.01N | | |
|---|---|---|
| NaOH | | Observations |
| Added, μL | pH | (Naked Eye) |
| 0 | 5.92 | Cloudy |
| 50 | 6.04 | Cloudy |
| 85 | 6.15 | Cloudy |
| 150 | 6.31 | Cloudy |
| 175 | 6.36 | Cloudy |
| 195 | 6.40 | Cloudy, optical image taken |
| 250 | 6.44 | Cloudy |
| 300 | 6.55 | Cloudy |
| 350 | 6.64 | Less cloudy, over time (5-10 minutes), |
| | | cloudiness is clearing, |
| | | optical image taken |
| 410 | 6.72 | Almost clear, in about 10 minutes |
| | | appeared clear, optical |
| | | image taken |
| 455 | 6.80 | Clear, optical image taken |

In FIG. 8A, the optical images show that at pH 6.40, drug delivery particles remained insoluble. At pH 6.64, drug delivery particles were beginning to swell and dissolve. At pH 6.72, optical imaging showed the majority of particles were dissolved and a small portion of swelled particles remained. At pH 6.80, optical imaging showed mostly dissolved particles and a couple of large aggregates consisting of swelled and shapeless particles. Aggregates appeared to dissolve over time, after approximately 20 minutes at pH 6.80. The study was repeated using drug delivery particles that were recently manufactured. After approximately one week of storage at 4° C., 700 μL of sample 817-83-1 was dispensed into a 1 dram vial. 0.02 N NaOH was added incrementally using a 250 μL Hamilton syringe fitted with a 27 gauge needle. Additions of NaOH were at least 2 minutes apart. 0.02 N NaOH was used in this study to minimize any potential dilution effect. Table 13 details the data collected in the study. FIG. 8A depicts the optical images.

TABLE 13

| 0.02N | | |
|---|---|---|
| NaOH | | Observations |
| Added, μL | pH | (Naked Eye) |
| 0 | 5.90 | Cloudy |
| 15 | 6.00 | Cloudy |
| 40 | 6.12 | Cloudy |
| 55 | 6.21 | Cloudy |
| 75 | 6.31 | Cloudy |
| 100 | 6.41 | Cloudy, optical image taken |
| 130 | 6.48 | Less cloudy, over time |
| | | (greater than 5 minutes), |
| | | cloudiness is clearing |
| 150 | 6.56 | Less cloudy, optical image taken |
| 175 | 6.65 | Some haziness remained, |
| | | after about 10 minutes |
| | | appeared clear |
| 190 | 6.74 | Almost clear, optical image taken |
| 205 | 6.85 | Clear, optical image taken |

In FIG. 8A the optical images show that at pH 6.41, drug delivery particles remained insoluble. At pH 6.56, drug delivery particles were swelling. At pH 6.74, optical imaging showed the majority of particles were dissolved. At pH 6.85, optical imaging showed mostly dissolved particles and a small amount of particle debris. Based on the experimental data, there were small differences between the drug delivery particles manufactured and stored for approximately one week and drug delivery particles manufactured and stored for approximately one year. The drug delivery particles started to swell when exposed to pH 6.50 to 6.55. The majority of drug delivery particles were dissolved when exposed to pH 6.65 to 6.74. The drug delivery particles were almost completely dissolved when exposed to pH 6.85 or higher.

Kinetics of Particle Dissolution by Optical Microscopy

Particle Fabrication:

Drug delivery particles were manufactured. First, a series of stock solutions were prepared. A homogeneous aqueous solution of 5 wt % PMMA-co-PMAA (˜125 kDa, MMA:MAA=2:1 molar ratio. Eudragit® S100, Evonik Industries) was made by dissolving the polymer at a pH of approximately 8. A second homogeneous aqueous solution of 5 wt % PMMA-co-PMAA (Lot 831-30, Example 5) was made in the same fashion. A homogeneous aqueous solution of 15 wt % PVP (2.5 kDa, Polysciences, Inc.) was prepared. The concentration of the Hib-TT solution was 897 μg/mL. The following volumes were combined to produce a particle stock solution: 1.932 mL PMMA-co-PMAA, 0.644 mL PVP, and 1.424 mL Hib-TT. Based on solid components the particle stock solution had the following weight percent ratio: 49.67 wt % PMMA-co-PMAA, 49.67 wt % PVP, and 0.66 wt % Hib-TT. A particle stock solution was produced with each of Eudragit® S100 and [831-30]. The resulting stock solutions were cast at room temperature onto raw PET film using a #13 Mayer rod. To form drug delivery particles, the film was laminated against a 6 μm donut PRINT® (Liquidia Technologies, Inc, Morrisville, N.C.) mould using a bench laminator. The drug delivery particles were removed from the film by mechanical scraping under dry conditions (i.e. less than 30% relative humidity). The process was repeated with both stock solutions.

Transition of Particles:

For each set of particles, under dry conditions (10-30% relative humidity), approximately 90-100 mg of drug delivery particles were sprinkled onto 4 mL of rapidly stirring 0.2 M sodium succinate pH 3.5/PEG400, 50/50 by volume. The particles were stirred about five minutes. After about five minutes, 4×2 mL 200 mM sodium maleate pH aliquots were added to the suspension, with about one minute of stirring between each aliquot addition. The suspension was divided into six tubes, approximately 2 mL per tube. The suspension was pelleted by spinning at 17,000×g for at least 5 minutes at 4° C. The supernatant was removed and discarded. Each pellet was resuspended in 1 mL 200 mM sodium maleate pH 6.1 and two tubes were combined into one tube. The suspension was pelleted by spinning at 17,000×g for at least 5 minutes at 4° C. The supernatant was removed and discarded. The pellet was resuspended in 2 mL 200 mM sodium maleate pH 6.1. The suspension was pelleted by spinning at 12,000×g for approximately 5 minutes at 4° C. The supernatant was removed and discarded. The pellet was resuspended in 2 mL 10 mM sodium maleate pH 6.1. The particle suspension was diluted by combining 1 mL of the suspension with 1 mL sodium maleate pH 6.1.

The particle suspension was filtered through 41 μm nylon net in 10 mM sodium maleate pH 6.1. The filtrate was concentrated by spinning the suspension at 12,000×g for at least 3 minutes at 4° C. The concentrated filtrate was pelleted by spinning at 12,000×g for approximately 3 minutes at 4° C. The supernatant was removed and discarded. The pellet was resuspended in 2 mL 10 mM sodium maleate pH 6.1 for a wash. The particles were washed two more times for a total of three washes. The particles were pelleted at 12,000×g for approximately 3 minutes at 4° C. The supernatant was removed and discarded. The pellet was resuspended in 2 mL 10 mM sodium maleate/150 mM NaCl pH 6.1. The particles were pelleted at 12,000×g for approximately 3 minutes at 4° C. The supernatant was removed and discarded. The pellet was resuspended in 1 mL 10 mM sodium maleate/150 mM NaCl pH 6.1.

The suspensions were analyzed gravimetrically for particle concentration. The concentration for sample 841-12-1 (Eudragit® S100 containing particles) was 7.75 mg/mL. The concentration for sample 841-12-3 (Lot [831-30] containing particles) was 1.725 mg/mL. Suspensions were diluted or concentrated to a particle concentration of approximately 2 mg/mL.

50 μL of sample 841-12-1 and sample 841-12-3 were placed into individual vials and were stirred at approximately 300 rpm at room temperature. 0.02 N NaOH was added to each sample, 10 μL to 841-12-1 and 18 μL to 841-12-3, and time was started. At T=2, 4, 6, 8, and 10 minutes, 2 μL of the sample was removed and evaluated using optical microscopy. FIG. 8B depicts the images. Table 14 summarizes observations of the particles using microscopy.

TABLE 14

| Time, | | |
|---|---|---|
| minutes | 841-12-1 | 841-12-3 |
| 2 | Particles swelling | Particles swelling |
| | to ~10-15 μm | and losing shape |
| 4 | Particles swelling | Not observed |
| | to ~20 μm | |
| 6 | Shapeless particles, | Debris, mostly |
| | some debris | lost shape |
| 8 | Debris | Little debris no |
| | | particles observed |
| 10 | Dissolved with | Dissolved with |
| | little debris | little debris |

### Example 6—Thermostability of pH-Sensitive Particles

A study was conducted to evaluate the thermal stability, as measured by free Hib (via HPAEC-PAD) over time (T=0, 14, 68, and 86 days), for drug delivery particles stored at various temperatures (25, 37, 45° C.).

Sample 855-72-2: 1.1 mL 855-61-1 was combined with 1.1 mL 10 mM sodium maleate/300 mM NaCl/0.02% thimerosal pH 6.1 and 10.857 mL 10 mM sodium maleate/150 mM NaCl/0.01% thimerosal pH 6.1 to produce a final particle concentration of approximately 2.381 mg/mL having a calculated total Hib content of 20 μg/mL. The production of 855-61-1 was described in Example 5.

Sample 855-72-4: 2.4 mL 855-14 was combined with 2.4 mL 10 mM sodium maleate/300 mM NaCl/0.02% thimerosal pH 6.1 and 8.340 mL 10 mM sodium maleate/150 mM NaCl/0.01% thimerosal pH 6.1 to produce a final particle concentration of approximately 2.283 mg/mL having a calculated total Hib content of 20 μg/mL. The production of 855-14 was described in Example 5. Relative to 855-72-2 above, 855-72-4 contains particles fabricated from a PMMA-co-PMAA polymer matrix of different origin, and which were transitioned in 0.4M, as opposed to 0.2M, sodium maleate pH 6.1.

855-72-5: As control, a sample containing soluble Hib (i.e. no particles) was produced by combining 0.27 mL Hib-TT (902 μg/mL) with 11.910 mL 10 mM sodium maleate/150 mM NaCl/0.01% thimerosal pH 6.1.

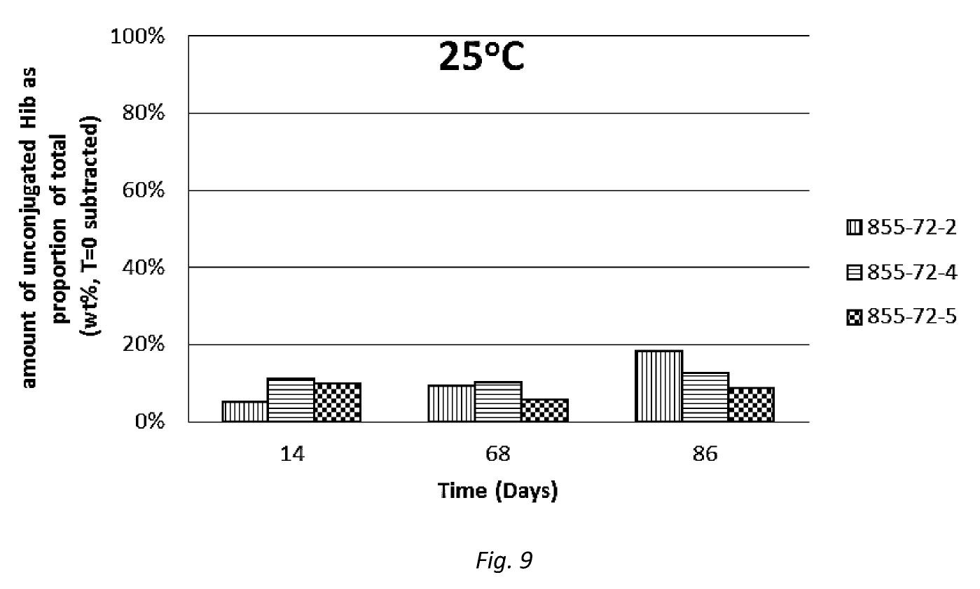

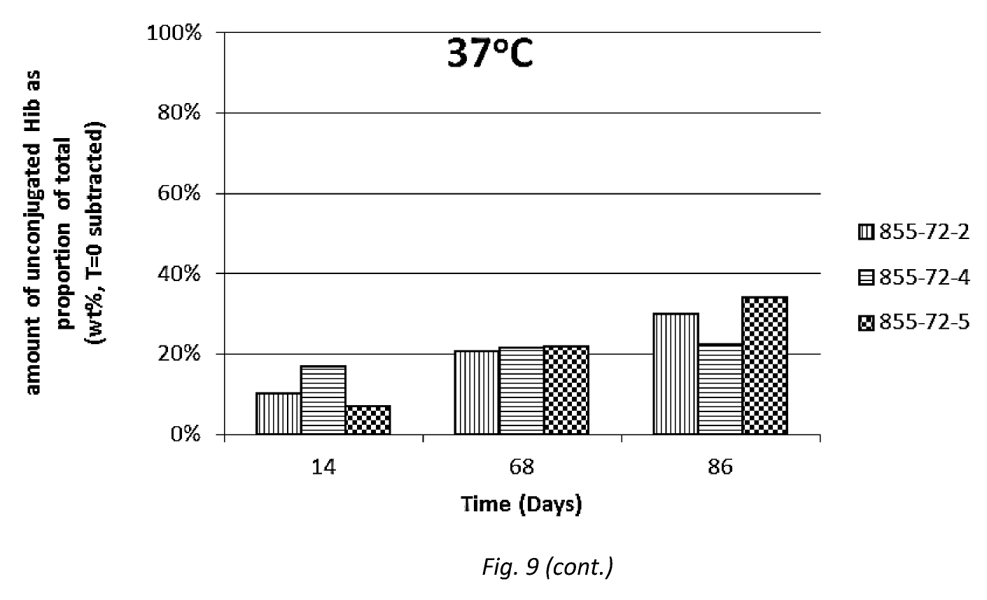

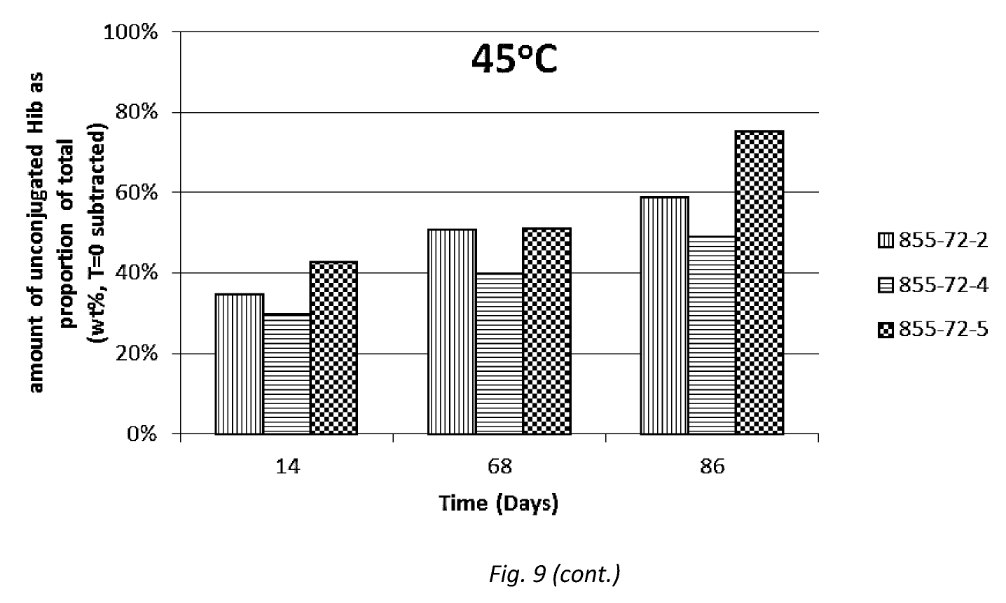

The samples were dispensed into vials in 0.65 mL aliquots and placed in storage at three temperatures: 25° C., 37° C., and 45° C. They were removed from storage at day 0, 14, 68, and 86 (approximately 0, 2, 10, 12 weeks) and analyzed for total Hib and free Hib in both the particles (pellet) and aqueous environment (supernatant) using HPAEC-PAD. Tables 15-17 show the proportion of Hib in free (unconjugated) form (pellet+supernatant, for particle-containing samples) at these timepoints, after subtraction of the respective free Hib amounts present at T=0. FIG. 9 graphically presents the information for timepoints 14, 68 and 86 days at 25, 37 and 45° C. There appears to be a protective effect of the particles on Hib-TT conjugate integrity at 37 and 45° C.

TABLE 15
855-72-2

| | 25° C. | | 37° C. | | 45° C. | |
|---|---|---|---|---|---|---|
| | | % Free Hib | | % Free Hib | | % Free Hib |
| | % Free | in Sample | % Free | in Sample | % Free | in Sample |
| | Hib in | (T = 0 | Hib in | (T = 0 | Hib in | (T = 0 |
| Interval | Sample | subtracted) | Sample | subtracted) | Sample | subtracted) |
| (days) | % | % | % | % | % | % |
| 0 | 15.79% | 0.00% | 15.79% | 0.00% | 15.79% | 0.00% |
| 14 | 21.09% | 5.30% | 26.21% | 10.42% | 50.53% | 34.74% |
| 68 | 25.10% | 9.31% | 36.58% | 20.79% | 66.66% | 50.87% |
| 86 | 34.22% | 18.42% | 45.89% | 30.10% | 74.65% | 58.86% |

TABLE 16
855-72-4

| | 25° C. | | 37° C. | | 45° C. | |
|---|---|---|---|---|---|---|
| | | % Free Hib | | % Free Hib | | % Free Hib |
| | % Free | in Sample | % Free | in Sample | % Free | in Sample |
| | Hib in | (T = 0 | Hib in | (T = 0 | Hib in | (T = 0 |
| Interval | Sample | subtracted) | Sample | subtracted) | Sample | subtracted) |
| (days) | % | % | % | % | % | % |
| 0 | 12.75% | 0.00% | 12.75% | 0.00% | 12.75% | 0.00% |
| 14 | 23.88% | 11.13% | 29.62% | 16.87% | 42.79% | 30.04% |
| 68 | 22.99% | 10.24% | 34.53% | 21.78% | 52.61% | 39.86% |
| 86 | 25.31% | 12.56% | 35.21% | 22.46% | 61.88% | 49.12% |

TABLE 17
855-72-5

| | 25° C. | | 37° C. | | 45° C. | |
|---|---|---|---|---|---|---|
| | | % Free Hib | | % Free Hib | | % Free Hib |
| | % Free | in Sample | % Free | in Sample | % Free | in Sample |
| | Hib in | (T = 0 | Hib in | (T = 0 | Hib in | (T = 0 |
| Interval | Sample | subtracted) | Sample | subtracted) | Sample | subtracted) |
| (days) | % | % | % | % | % | % |
| 0 | 8.43% | 0.00% | 8.43% | 0.00% | 8.43% | 0.00% |
| 14 | 18.39% | 9.96% | 15.55% | 7.11% | 51.35% | 42.92% |
| 68 | 14.29% | 5.85% | 30.29% | 21.85% | 59.57% | 51.14% |
| 86 | 17.11% | 8.67% | 42.46% | 34.03% | 83.84% | 75.14% |

### Example 7—In Vivo Study

An in vivo study was performed to demonstrate the ability of the pH-sensitive particles to protect Hib-TT cargo from deleterious interaction with the Infanrix Penta formulation during storage, whilst allowing the cargo to elicit an immune response following administration. An adult rat model was used.

Material and Methods:

Hib-TT-containing particles were added to Infanrix Penta to make a hexavalent DTPa-HepB-IPV-Hib composition. After a minimum of 4 week storage at 4° C., these compositions were administered to the rats in Groups 4 and 5, discussed below. 6 week old female adult Sprague-Dawley rats (Ico:OFA-SD) were divided into 5 groups (see below; n=20 per group). Immunisation with a one-tenth human dose, administered intramuscularly, occurred on days 0, 14, and 28. Animals were bled for serology on days 21 (“7PII”) and 35 (“7PIII”).

Group 1: Lyophilised Hib-TT (adsorbed on aluminium phosphate) was reconstituted with Infanrix Penta (=Infanrix Hexa) in a final volume of about 625 μL. 50 μL (1 μg/ml Hib dose) was administered extemporaneously (i.e. within 1 hour post-reconstitution).

Group 2: Lyophilised Hib-TT (Hiberix) was reconstituted with 625 μL of NaCl 150 mM (1 μg/mL Hib dose in 50 μL) and co-administered (at different sites) with Infanrix Penta (50 μL).

Group 3: Hib-TT-containing particles 841-57-1 were diluted in NaCl 150 mM 10 mM maleate buffer pH 6.1 to a Hib-TT concentration of 20 μg/mL. 55 μL of the diluted sample was administered and Infanrix Penta (50 μL) was co-administered (at a different site).

Group 4: 55 μL of Hib-TT-containing particles in Infanrix Penta 841-57-2 were administered after storage at 4° C. for 4 weeks.

Group 5: 55 μL of Hib-TT-containing particles in Infanrix Penta 700-66 were administered after storage at 4° C. for 16 months.

Serology Analysis for Hib Antigen (PRP):

Sera from all rats were individually collected seven days after the second (7PII) or third (7PIII) immunization and tested for the presence of Haemophilus influenzae type b polyribosyl-ribitol-phosphate (PRP)-specific IgG antibodies according to the following protocol. 96-well plates were coated with tyraminated PRP (1 μg/ml) in a carbonate-bicarbonate buffer (50 mM) and incubated overnight at 4° C. Rat sera were diluted at 1/10 in PBS-Tween 0.05% and serially diluted in the wells from the plates (12 dilutions, step 1/2). An anti-Rat IgG (H+L) polyclonal antibody coupled to the peroxidase was added (1/5000 dilution). Colorimetric reaction was observed after the addition of the peroxidase substrate (OPDA), and stopped with HCL (1M) before reading by spectrophotometry (wavelengths: 490-620 nm). For each serum tested and standard added on each plate, a 4-parameter logistic curve was fit to the relationship between the OD and the dilution (Softmaxpro). This allowed the derivation of each sample titer expressed in STD titers. The statistical method employed was an Analysis of Variance (ANOVA) on the log 10 values with 2 factors (group and TP) using a heterogeneous variance model i.e. identical variances were not assumed for the different levels of the factor. The interaction between the 2 factors was tested; results are provided by level of the second factor since interactions appeared to be qualitative in nature. Estimates of the geometric mean ratios between groups and their 95% confidence intervals (CI) were obtained using back-transformation on log 10 values. Adjustment for multiplicity was performed using Tukey's method. Multiplicity adjusted 95% confidence intervals were provided.

Results of Hib Serology:

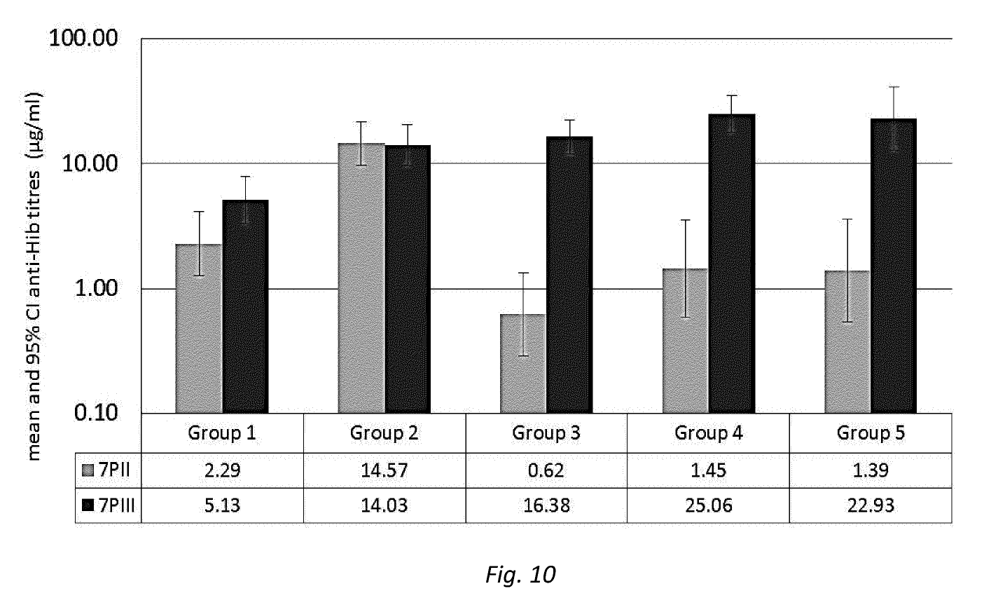

The Hib serology results are shown in FIG. 10. At both 7PII and 7PIII Groups 1 and 2 were statistically significantly different, validating the ability of the experiment to demonstrate the occurrence of interference between the Hib-TT and the Infanrix Penta (apparent as reduced anti-Hib immune response).

At 7PIII, Group 1 (extemporaneous reconstitution of lyophilised Hib-TT with Infanrix Penta=Infanrix Hexa) was statistically significantly different from Groups 2-5 in which the Hib-TT was co-administered (i.e. at separate sites, not mixed; Groups 2 and 3) with Infanrix Penta and/or was present within a drug delivery particle (Groups 3-5). Hence by mixing Hib-TT-containing particles with Infanrix Penta, a reduction in interference was observed. Note that the lower anti-Hib immune response in Group 1 occurs on extemporaneous mixing, i.e. even in the absence of storage of the mixture for any extended duration.

At 7PIII no significant difference was detected between co-administration (not mixing) of Hib-TT-containing particles with Infanrix Penta (Group 3) and administration of particles which had been mixed with Infanrix Penta for 4 weeks (Group 4). This demonstrates that the particle prevents or minimises deleterious interaction between the Hib-TT and Infanrix Penta during storage, while allowing access of the Hib-TT to the immune system once administered.

Statistical equivalence was detected between Groups 2 and 3 at 7PIII, showing that the formulation of Hib-TT within a particle does not impair its ability to induce an immune response once administered. Groups 4 and 5 were also shown to be equivalent at 7PIII, suggesting no or minimal loss of Hib-TT immunogenicity, and no or minimal loss of Hib-TT from the particles, during the storage period from 4 weeks up to more than one year, i.e. Hib-TT in the particles is stable in aqueous formulation.

1-113. (canceled)

114. A plurality of pH-sensitive drug delivery particles comprising a biologically active cargo within a matrix, wherein said particles are triggered to release said cargo by being present in an aqueous environment having a higher pH relative to the pH of an aqueous environment in which said particles are formulated and maintained prior to being so triggered.

115. The plurality of particles accordingly to claim 114, wherein said particles are intact at a sub-physiological pH, and wherein on subjecting said particles to a trigger physiological pH said particles are substantially or completely dissolved or degraded within 24 hours or less.

116. The plurality of particles according to claim 114, wherein said matrix is insoluble in an aqueous environment at a sub-physiological pH, and wherein said matrix is soluble in an aqueous environment at a trigger physiological pH.

117. The plurality of particles according to claim 116, wherein said sub-physiological pH is at or below 6.8, 6.7, 6.6, 6.5, 6.4, 6.3, 6.2, 6.1 or 6.0.

118. The plurality of particles according to claim 117, wherein said polymeric matrix comprises (poly(methyl methacrylate)-co-poly(methacrylic acid) copolymer (PMMA-co-PMAA copolymer); poly(glutamic acid)-co-poly(lysine); zwitterionic hetero- or homo-poly(amino acids); carboxymethyl chitosan; hypromellose phthalate; hypromellose acetate succinate; or an acrylate co-polymer represented by the general formula (1) wherein: 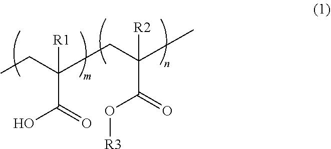
R1 represents hydrogen or methyl, R2 represents hydrogen or methyl, and R3 represents methyl, ethyl, propyl, isopropyl, n-butyl, iso-butyl, tert-butyl, sec-butyl, phenyl, or benzyl.

119. The plurality of particles according to claim 117, wherein said polymeric matrix comprises PMMA-co-PMAA copolymer, wherein the molar ratio of methyl methacrylate (MMA) monomer to methacrylic acid (MAA) monomer in the copolymer is in the range 1:1-4:1, in particular 1.5-2:1.

120. The plurality of particles according to claim 117, wherein said PMMA-co-PMAA copolymer has a weight average molecular weight (Mw) in the range 1-200 kDa, such as in the range 50-60 kDa or 35-45 kDa or 22-28 kDa or 8-12 kDa, such as a weight average molecular weight (Mw) of 10, 25, 40, 55 or 125 kDa.

121. The plurality of particles according to claim 114, wherein said matrix is polymeric.

122. The plurality of particles according to claim 121, wherein said polymeric matrix is biocompatible or biodegradable or bioresorbable or excretable, in or from the human body.

123. The plurality of particles according to claim 114, wherein said cargo comprises an oligo/polysaccharide antigen, optionally conjugated to a carrier protein such as tetanus toxoid, fragment C of tetanus toxoid, diphtheria toxoid, CRM197 or another non-toxic mutant of diphtheria toxin, protein D of non-typeable Haemophilus influenzae, outer membrane protein complex (OMPC) of Neisseria meningitidis, pneumococcal PhtD, pneumococcal pneumolysin, exotoxin A of Pseudomonas aeruginosa (EPA), detoxified haemolysin of Staphylococcus aureus, detoxified adenylate cyclase of Bordetella sp, detoxified Escherichia coli heat labile enterotoxin, or cholera toxin subunit B (CTB) or detoxified cholera toxin.

124. The plurality of particles according to claim 114, wherein said particles are moulded resulting in a precisely predetermined size and shape, in particular a donut shape.

125. The plurality of particles according to claim 114, wherein the longest axis of said particles is between about 1-10 μm, in particular between 5-7 μm such as 6 μm.

126. The plurality of particles according to claim 114, wherein at least one axis is less than 200 nm, such as less than 100 nm, and said particle is sterile filterable.

127. The plurality of particles according to claim 114, wherein said particles are for parenteral administration, such as intradermal or subcutaneous, in particular intramuscular administration.

128. A composition, particularly an immunogenic composition, comprising a plurality of particles as defined in claim 114 in an aqueous environment, optionally wherein said environment is sterile.

129. The composition or immunogenic composition according to claim 128, wherein said composition or immunogenic composition is a vaccine.

130. An immunogenic composition comprising a plurality of particles as defined in claim 114 in an aqueous environment, wherein:
a. said particles comprise a Hib-TT or Hib-CRM197 cargo homogeneously dispersed within a matrix comprising PMMA-co-PMAA; and
b. said aqueous environment comprises diphtheria toxoid, tetanus toxoid and acellular pertussis antigens adsorbed onto aluminium hydroxide, and optionally HBsAg and IPV.

131. The plurality of particles according to claim 114 for use in medicine, in particular human medicine.

132. A method for making a plurality of drug delivery particles comprising a biologically active cargo within a matrix, comprising the steps of:
a. at least partially deprotonating a polymer, which is insoluble in its protonated state, in an aqueous environment such that the polymer has a net negative charge and is soluble in said aqueous environment;
b. combining said polymer with said cargo to produce a solution;
c. forming particles by moulding said solution and removing the aqueous environment.

133. A plurality of drug delivery particles obtainable or obtained by the method of claim 132.
